SEQUENCE LISTING (4 sequences)

SEQ ID NO: 1 - DNA, 20 bp, synthetic construct
  misc_feature 1..20: note = 16S rRNA forward primer
  source 1..20: mol_type = other DNA
ctcttggaaacgggtggtaa

SEQ ID NO: 2 - DNA, 20 bp, synthetic construct
  misc_feature 1..20: note = 16S rRNA reverse primer
  source 1..20: mol_type = other DNA
tgctcccaatcaaaagcggt

SEQ ID NO: 3 - DNA, 20 bp, synthetic construct
  misc_feature 1..20: note = vaginolysin (set 1) forward primer
  source 1..20: mol_type = other DNA
ctcgcatgcagtacgattct

SEQ ID NO: 4 - DNA, 20 bp, synthetic construct
  misc_feature 1..20: note = vaginolysin (set 1) reverse primer
  source 1..20: mol_type = other DNA
tctggtgcatcaacgcttac

(19) United States
(12) Patent Application Publication
(10) Pub. No.: US 2023/0398178 A1
(43) Pub. Date: Dec. 14, 2023

(54) METHODS AND COMPOSITIONS FOR TREATING OR PREVENTING A VAGINAL INFECTION OF GARDNERELLA VAGINALIS

(71) Applicant: Massachusetts Institute of Technology, Cambridge, MA (US)

(72) Inventors: Katharina Ribbeck, Cambridge, MA (US); Laura L. Kiessling, Cambridge, MA (US); Caroline Andrea Werlang, Cambridge, MA (US); Spencer D. Brucks, Claremont, CA (US); Liubov Yakovlieva, Cambridge, MA (US); Kelsey Morgan Wheeler, Cambridge, MA (US)

(21) Appl. No.: 18/334,253

(22) Filed: Jun. 13, 2023

Related U.S. Application Data
(60) Provisional application No. 63/351,552, filed on Jun. 13, 2022.

(51) Int. Cl. A61K 38/17; A61K 31/7004; A61K 35/747; A61K 31/4164; A61K 47/64; A61P 31/04; A61K 31/7008; A61K 47/58

(52) U.S. Cl. CPC A61K 38/1735; A61K 31/7004; A61K 35/747; A61K 31/4164; A61K 47/64; A61P 31/04; A61K 31/7008; A61K 47/58

(57) ABSTRACT

Methods of treating or preventing a vaginal infection of Gardnerella vaginalis in a subject involve administering to the subject an effective amount of a composition. The composition can include one or more of galactose, D-fucose, or mucin 5B. The composition can include one or more of galactose, N-acetyl D-galactosamine, N-acetyl D-glucosamine, D-fucose, or mucin 5B bonded to a surface, such as a polymer surface.

### RELATED APPLICATION

This application claims the benefit of U.S. Provisional Application No. 63/351,552, filed on Jun. 13, 2022. The entire teachings of the above application are incorporated herein by reference.

### GOVERNMENT SUPPORT

This invention was made with government support under EB017755 awarded by the National Institutes of Health. The government has certain rights in the invention.

### INCORPORATION BY REFERENCE OF MATERIAL IN XML

This application incorporates by reference the Sequence Listing contained in the following eXtensible Markup Language (XML) file being submitted concurrently herewith:
a) File name: 00502380001_Sequence_Listing.xml; created Jun. 13, 2023, 6,179 Bytes in size.

### BACKGROUND

Bacterial vaginosis is an imbalance in the vaginal microbiota that affects up to a third of people with vaginas throughout the world (FIG. 1A).^{1,7-9} Its symptoms can vary widely, and it is characterized into four community state types (CSTs) with varying severity. CSTI is a Lactobacillus-dominant community, generally the species L. crispatus, and is considered the “healthy” state. In CSTI communities, beneficial Lactobacilli like L. crispatus metabolize glycogen in the vaginal environment to produce lactic acid, maintaining the vagina at a characteristically healthy pH of 4, and preventing the proliferation of other organisms.¹⁰⁻¹³ CSTII is still Lactobacillus dominant, but contains a high relative proportion of Lactobacillus iners.¹⁴⁻¹⁶ CSTIII and CSTIV communities are considered “BV” and contain higher proportions of a diverse set of anaerobic bacteria, including the genera Prevotella¹⁷, Atopobium¹⁸, and Gardnerella¹⁹, and are characterized by a raised pH level.²⁰⁻²² A variety of factors, including host genetic factors and lifestyle could potentially affect a person's CST, and it is suggested that the digestive tract is the origin of vaginal bacteria.²³ However, the exact cause of BV is unknown. Many people are able to host low numbers of potentially pathogenic organisms²⁴, maintaining a symptom-free CSTI or CSTII state, suggesting that some host factors may be able to contribute to domestication of opportunistic pathogens such as Gardnerella vaginalis.²⁵

The current standard treatment for BV is oral or intravaginally applied antibiotics, which effectively clear out the potentially pathogenic anerobic bacteria characteristic of CSTIII and CSTIV communities; unfortunately, these broad-spectrum antibiotic treatments also target beneficial commensal Lactobacilli.⁵ As many as 30% of patients who undergo standard antibiotic treatments have a recurrence of BV,²⁶ and the diminishment of beneficial Lactobacilli leaves patients vulnerable to secondary infections like vulvovaginal candidiasis. Novel methods of treatment, which involve introducing new strains of probiotic Lactobacilli have been proposed.²⁷ A small five-person pilot study of vaginal microbiota transplants found that treating BV patients with antibiotics and then inoculating them with strains from healthy donors was able to lead to lasting conversion to CSTI communities in four of five patients.²⁸ While these treatments showed great promise, they were not successful for all patients in the cohort, indicating that further interventions may be needed.

### SUMMARY

This Summary introduces a selection of concepts in simplified form that are described further below in the Detailed Description. This Summary neither identifies key or essential features, nor limits the scope, of the claimed subject matter.

Described herein is a method of treating or preventing a vaginal infection of Gardnerella vaginalis in a subject. The method can include administering to the subject an effective amount of a composition comprising one or more of galactose, D-fucose, or mucin 5B. In some instances, the composition includes galactose. In some instances, the composition includes D-fucose. In some instances, the composition includes mucin 5B. In any of the foregoing methods, the galactose, N-acetyl D-galactosamine, N-acetyl D-glucosamine, D-fucose, or mucin 5B can administered in an amount from 0.1 wt % to 50 wt %.

Described herein is a method of treating or preventing a vaginal infection of Gardnerella vaginalis in a subject. The method can include administering to the subject an effective amount of a composition comprising one or more of galactose, N-acetyl D-galactosamine, N-acetyl D-glucosamine, D-fucose, or mucin 5B bonded to a surface. In some instances, galactose bonded to the surface. In some instances, N-acetyl D-galactosamine bonded to the surface. In some instances, N-acetyl D-glucosamine bonded to the surface. In some instances, D-fucose bonded to the surface. In some instances, mucin 5B bonded to the surface. In any of the foregoing methods, the surface can be a polymer, a silk fibroin (SF), a norbornene polymer, a star polymer, or a dendrimer. In any of the foregoing methods, the galactose, D-fucose, or mucin 5B can be administered in an amount from 0.1 wt % to 50 wt %.

In any of the foregoing methods, the method can reduce the concentration of Gardnerella vaginalis in the vagina of the subject. The method can reduce expression of vaginolysin by Gardnerella vaginalis in the vagina of the subject. The method can reduce concentration of vaginolysin in the vagina of the subject. The method can reduce mortality rates for endocervical cells of the subject. The method can reduce biofilm formation caused by the vaginal infection of Gardnerella vaginalis. The method can treat the vaginal infection of Gardnerella vaginalis. The method can prevent the vaginal infection of Gardnerella vaginalis. The subject can have bacterial vaginosis. In some instances, the subject has previously had at least three bacterial vaginosis infections.

Any of the foregoing methods can include diagnosing the subject with the vaginal infection of Gardnerella vaginalis.

In any of the foregoing methods, the composition can be formulated for topical or vaginal administration.

Any of the foregoing methods can include administering a Lactobacillus probiotic to the subject.

Any of the foregoing method can include administering an antibiotic, such as metronidazole, to the subject.

The following Detailed Description references the accompanying drawings that form a part this application, and which show, by way of illustration, specific example implementations. Other implementations may be made without departing from the scope of the disclosure.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-E: The mucin MUC5B reduces relative biofilm formation of G. vaginalis. (FIG. 1A) Canonically, healthy vaginal microbiomes are dominated by Lactobacillus crispatus. In general, when there is an overgrowth of non-Lactobacillus species, especially Gardnerella vaginalis, it can lead to a condition called bacterial vaginosis. This condition is highly prevalent worldwide. (FIG. 1B) G. vaginalis can be grown into biofilms in polystyrene plates. (FIG. 1C) MUC5B, the predominant mucin found in cervicovaginal fluid, can be isolated from human saliva and added into in vitro experiments. (FIG. 1D) To test whether this may be due to an effect of mucin's glycans, we assessed the ability of its component monosaccharides, L-Fucose, galactose (Gal), GalNAc, GlcNAc, and NeuNAc, to prevent biofilm formation. Additionally, we tested other sugars, glucose (Glc) and D-Fucose (the stereoisomer of L-Fuc; also known as 6-deoxy-D-galactose). We found that galactose and D-Fuc both significantly reduced proportional biofilm formation in G. vaginalis 14018 24 hour biofilms in NYCIII media. (FIG. 1E) It is important to note that MUC5B reduced the number of biofilm cells relative to the total population, but did not limit the growth of the bacteria. On the other hand, galactose and D-fucose both reduced proportional biofilm formation and also reduced the overall growth of these bacteria in NYCIII media (which contains 1% glucose).

FIGS. 2A-C: Biofilm formation (FIG. 2A) and total growth (FIG. 2B) of G. vaginalis 49145 were reduced in the presence of D-fucose. (FIG. 2C) D-Fucose is highly similar to D-Galactose, a natural sugar, but is lacking a hydroxyl group on its sixth carbon. Its structural similarity may enable it to act as an inhibitor of normal galactose utilization.

FIG. 3A-E: Mucin and D-fucose can prevent G. vaginalis killing of End1 cervical cells in vitro, potentially by reducing the expression of vaginolysin. (FIG. 3A) The End1 cell line of endocervical cells does not secrete gel-forming mucins. To assess the protective ability of MUC5B, it can be added to apical surface of the cells. The cells can then be challenged with G. vaginalis, and the percentage of endocervical cell death can be recorded. (FIG. 3B) Addition of MUC5B to the co-culture results in higher survival rates for the endocervical cells when challenged with G. vaginalis 49145 or 14018. (FIG. 3C) Treatment with D-Fucose protects against killing by G. vaginalis 49145 and 14018. Carboxymethyl cellulose (CMC), a physical barrier, did not provide protection. (FIG. 3D) A key mechanism by which G. vaginalis kills endocervical cells is by secreting the cholesterol-dependent cytolysin vaginolysin (vly). Control experiments (FIGS. 4A-D) confirm that strains of G. vaginalis that lack vaginolysin do not kill endocervical cells. We measured vaginolysin expression by RT-qPCR. We found that compared to media, G. vaginalis 14018 and 49145 expressed less vaginolysin in the presence of MUC5B and D-fucose. (FIG. 3E) G. vaginalis showed reduced expression of vaginolysin over time in the presence of MUC5B, through exponential phase growth.

FIGS. 4A-D: Grafting mucin sugars onto polymers creates specific antimicrobial polymers. (FIG. 4A) SF(S)-Gal, SF(S)-GalNAc, and SF(S)-GlcNAc are toxic to G. vaginalis 14018, but SF(S)-Glc and SF(S)—NeuNAc are not. (FIG. 4B) A similar effect was seen when Gal was grafted onto a norbornene backbone.⁴² (FIG. 4C) The silk polymers and the norbornene polymers were not toxic to Lactobacillus crispatus JVV01, a commensal organism. (FIG. 4D) A combination therapy of mucin-inspired glycopolymers with a live Lactobacillus probiotic could restore a stable vaginal microbiome in people with persistent bacterial vaginosis.

FIG. 5: Norbornene glycopolymers follow the same pattern of glycan and organism specificity as silk-derived glycopolymers. Left: Galactose displaying polymers are toxic to G. vaginalis 14018, but glucose displaying polymers do not have an effect. Right: Galactose displaying polymers do not harm L. crispatus JV001.

FIG. 6: Strains of G. vaginalis without vaginolysin do not kill endocervical cells. Presence or absence of the vaginolysin gene was confirmed by PCR of the flanking regions. Strain G. vaginalis JCP 7275 does not encode a gene for vaginolysin, and does not kill endocervical cells.

FIGS. 7A-B. MUC5B mucin and its specific monosaccharides disrupt established biofilms of Gardnerella vaginalis. FIG. 7A: Colony-forming units counts for biofilm and supernatant fractions of Gardnerella vaginalis cultures under different conditions. FIG. 7B: Biofilm dispersion mechanism of MUC5B mucin and D-Gal/D-Fuc.

FIGS. 8A-C. Galactose and D-Fucose potentiate the action of antibiotic metronidazole in a minimum biofilm eradication assay. Metronidazole concentration series alone or in combination with Galactose/D-Fucose (at 0.2%) in biofilm eradication assay. Cell counts are shown relative to the starting biofilm established over 24 h of culturing.

### DETAILED DESCRIPTION

A description of example embodiments follows.

Reference numbers in superscripts herein refer to the corresponding literature listed in the attached Bibliography which forms a part of this Specification, and the literature is incorporated by reference herein.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly-used dictionaries, should be interpreted as having a meaning that is consistent with their meaning in the context of the relevant art and/or as otherwise defined herein.

The terminology used herein is for the purpose of describing particular embodiments only and is not intended to be limiting.

When introducing elements disclosed herein, the articles “a,” “an,” “the,” and “said” are intended to mean that there are one or more of the elements. Further, the one or more elements may be the same or different.

Throughout this specification and the claims which follow, unless the context requires otherwise, the word “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, “consisting of” excludes any element, step, or ingredient not specified in the claim element. When used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

It should be understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

Compounds described herein include those described generally, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the relevant contents of which are incorporated herein by reference.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by reference herein for its chemical structure names and rules on naming chemical structures. Optionally, a name of a compound may be generated using a chemical naming program (e.g., CHEMDRAW®, version 17.0.0.206, PerkinElmer Informatics, Inc.).

The phrase “pharmaceutically acceptable” means that the substance or composition the phrase modifies is, within the scope of sound medical judgment, suitable for use in contact with the tissues of humans and lower animals without undue toxicity, irritation, allergic response and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the compounds described herein include salts derived from suitable inorganic and organic acids, and suitable inorganic and organic bases.

Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

Compounds described herein can also exist as “solvates” or “hydrates.” A “hydrate” is a compound that exists in a composition with one or more water molecules. A hydrate can include water in stoichiometric quantities, such as a monohydrate or a dihydrate, or can include water in random amounts. A “solvate” is similar to a hydrate, except that a solvent other than water, such as methanol, ethanol, dimethylformamide, diethyl ether, or the like replaces water. Mixtures of such solvates or hydrates can also be prepared. The source of such solvate or hydrate can be from the solvent of crystallization, inherent in the solvent of preparation or crystallization, or adventitious to such solvent.

Compounds disclosed herein may exist as stereoisomers. For example, compounds disclosed herein may have asymmetric centers, chiral axes, and chiral planes (e.g., as described in: E. L. Eliel and S. H. Wilen, Stereo-chemistry of Carbon Compounds, John Wiley & Sons, New York, 1994, pages 1119-1190), and occur as racemates, racemic mixtures, or as individual diastereomers or enantiomers.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

“Treating” or “treatment,” as used herein, refers to taking steps to deliver a therapy to a subject, such as a mammal, in need thereof (e.g., as by administering to a mammal one or more therapeutic agents). “Treating” or “treatment” includes inhibiting the disease or condition (e.g., as by slowing or stopping its progression or causing regression of the disease or condition), and relieving the symptoms resulting from the disease or condition. The term “treating” or “treatment” refers to the medical management of a subject with the intent to improve, ameliorate, stabilize (i.e., not worsen), prevent or cure a disease, pathological condition, or disorder—such as the particular indications exemplified herein. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

“Administering” or “administration,” as used herein, refers to providing a compound, composition, or pharmaceutically acceptable salt thereof described herein to a subject in need of treatment or prevention.

“A therapeutically effective amount” or “an effective amount” refers to an amount effective, at dosages and for periods of time necessary, to achieve a desired therapeutic or biological result (e.g., treatment, healing, inhibition or amelioration of physiological response or condition, etc.). Non-limiting examples of desired therapeutic or biological results include disruption of biofilm formation, growth, and/or maintenance, for example, at or proximate to the surface of an implanted medical device. Effective reductions of signs and/or symptoms associated with infection can be determined by one or more suitable means in the art.

The full therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of an individual, e.g., a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. For example, suitable dosages can be from about 0.001 mg/kg to about 100 mg/kg, from about 0.01 mg/kg to about 100 mg/kg, from about 0.01 mg/kg to about 10 mg/kg, or from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

### Methods of the Disclosure

In one aspect, the disclosure provides methods of treating or preventing bacterial vaginosis in a subject in need thereof. The method can include administering to the subject an effective amount of galactose (Gal), N-acetylgalactosamine (GalNAc), N-acetylglucosamine (GlcNAc), or D-fucose (D-Fuc), or a pharmaceutically acceptable salt of any thereof.

In some embodiments, the subject has a vaginal infection of Gardnerella vaginalis.

“Virulence,” as used herein, refers to a phenotypic state of Gardnerella vaginalis associated with infection that may harm its host, for example, the host's endocervical cells. In some embodiments, the method reduces the quantity of a population of Gardnerella vaginalis in the vagina, reduces mortality rates for endocervical cells, reduces expression of vaginolysin in the vagina, or a combination thereof.

In some embodiments, a method disclosed herein reduces expression of vaginolysin in the vagina by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-70%, 45-70%, 45-65%, 50-65% or 50-60%. In some embodiments, a method reduces expression of vaginolysin in the vagina by at least about 30%.

Expression of vaginolysin in the vagina can be determined by a person of ordinary skill using methods known in the art, for example, by quantitative reverse transcription polymerase chain reaction (RT-qPCR), or, alternatively, at the RNA level using RNA sequencing (RNA-Seq) or a microarray.

In some embodiments, attenuating virulence of Gardnerella vaginalis includes inhibiting or reducing biofilm formation. “Biofilm,” as used herein, refers to a structured community of microbial cells that is adherent to a surface. In some embodiments, a method reduces biofilm formation by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-85%, 30-80%, 35-80%, 35-75%, 40-75%, 40-70%, 45-70%, 45-65%, 50-65% or 50-60%. In some embodiments, a method reduces biofilm formation by at least about 30%.

In another aspect, described herein are methods of inhibiting formation of a biofilm on a surface. The methods include contacting the surface with a monosaccharide (e.g., a therapeutically effective amount of a monosaccharide). The methods can also include contacting the surface with a composition that includes a glycopolymer of a monosaccharide (e.g., a therapeutically effective amount of a composition that includes a glycopolymer of a monosaccharide). The monosaccharide can be galactose (Gal), N-acetylgalactosamine (GalNAc), N-acetylglucosamine (GlcNAc), or D-fucose (D-Fuc). In some instances, the method can include contact the surface with mucin 5B (MUC5B).

As used herein, “subject” includes humans, domestic animals, such as laboratory animals (e.g., dogs, monkeys, pigs, rats, mice, etc.), household pets (e.g., cats, dogs, rabbits, etc.) and livestock (e.g., pigs, cattle, sheep, goats, horses, etc.), and non-domestic animals. In some embodiments, a subject is a human.

“Subject in need thereof,” as used herein, refers to a subject (e.g., a mammalian subject such as a human) diagnosed with or suspected of having a vaginal infection of Gardnerella vaginalis. “Subject in need thereof” includes those subjects who already have the undesired physiological change or disease as well as those subjects prone to have the physiological change or disease. “Subject in need thereof” can also refer to a subject diagnosed with or suspected of having bacterial vaginosis.

The administration of the compounds (agents, salts, etc.) and compositions typically occurs by topical or vaginal administration at or near the site of infection.

In some embodiments, a composition is provided in a liquid form. In some embodiments, a composition comprises a dose of from about 0.1 g/liter to about 50 g/liter, for example, about: 0.1 g/liter, 0.2 g/liter, 0.3 g/liter, 0.4 g/liter, 0.5 g/liter, 0.6 g/liter, 0.7 g/liter, 0.8 g/liter, 0.9 g/liter, 1 g/liter, 2 g/liter, 3 g/liter, 4 g/liter, 5 g/liter, 6 g/liter, 7 g/liter, 8 g/liter, 9 g/liter, 10 g/liter, 11 g/liter, 12 g/liter, 13 g/liter, 14 g/liter, 15 g/liter, 16 g/liter, 17 g/liter, 18 g/liter, 19 g/liter, 20 g/liter, 21 g/liter, 22 g/liter, 23 g/liter, 24 g/liter, 25 g/liter, 26 g/liter, 27 g/liter, 28 g/liter, 29 g/liter, 30 g/liter, 31 g/liter, 32 g/liter, 33 g/liter, 34 g/liter, 35 g/liter, 36 g/liter, 37 g/liter, 38 g/liter, 39 g/liter, 40 g/liter, 41 g/liter, 42 g/liter, 43 g/liter, 44 g/liter, 45 g/liter, 46 g/liter, 47 g/liter, 48 g/liter, 49 g/liter, 50 g/liter, 60 g/liter, 70 g/liter, 80 g/liter, 90 g/liter, 100 g/liter, 150 g/liter, 200 g/liter, 250 g/liter, 300 g/liter, 350 g/liter, 400 g/liter, 450 g/liter, or 500 g/liter.

In some embodiments, a composition comprises a dose of from about 0.1 wt % to 50 wt %, for example about 0.1 wt %, 0.2 wt %, 0.3 wt %, 0.4 wt %, 0.5 wt %, 0.6 wt %, 0.7 wt %, 0.8 wt %, 0.9 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, or 50 wt %. In some embodiments, the composition comprises a dose of from about 0.1 wt % to 10 wt %. In some embodiments, the composition comprises a dose of from about 0.1 wt % to 5 wt %. In some embodiments, the composition comprises a dose of from about 0.1 wt % to 3 wt %. In some embodiments, the composition comprises a dose of from about 0.1 wt % to 2 wt %. In some embodiments, the composition comprises a dose of from about 0.1 wt % to 1 wt %. In some embodiments, the composition comprises a dose of from about 0.1 wt % to 0.5 wt %. In some embodiments, the composition comprises a dose of from about 0.5 wt % to 10 wt %. In some embodiments, the composition comprises a dose of from about 0.5 wt % to 5 wt %. In some embodiments, the composition comprises a dose of from about 0.5 wt % to 2 wt %. In some embodiments, the composition comprises a dose of from about 0.5 wt % to 1 wt %.

In some embodiments, a composition is provided in a dried form.

In some embodiments, a composition is provided in a gel form.

Administration of a compound, composition, or pharmaceutically acceptable salt described herein may be in conjunction with another active ingredient (e.g., an antibiotic, such as metronidazole), for example, simultaneously in the same composition, simultaneously in different dosage forms, or sequentially. A compound, composition, or pharmaceutically acceptable salt described herein and another active ingredient may be formulated in a single combination, multiple combinations, or separate compositions.

The other active ingredient (e.g., an antibiotic, such as metronidazole) can be administered in any suitable manner, e.g., by parenteral or nonparenteral administration, including by aerosol inhalation, injection, infusions, ingestion, transfusion, implantation or transplantation. For example, the other active ingredient described herein may be administered to a subject trans-arterially, intradermally, subcutaneously, intratumorally, by intramedullar administration, intranodally, intramuscularly, intravenously (e.g., through an IV drip or by intravenous (i.v.) injection), intranasally, intrathecally or intraperitoneally. In some embodiments, the administration is intravenous. In some embodiments, the administration is topical. In some embodiments, the administration is oral. In some embodiments, the administration is by injection, for instance, directly into a tissue, organ, or site of infection. In some embodiments, the administration is ex vivo. In some embodiments, other active ingredient is administered by routes such as oral, endobronchial, intrathecal, intracisternal, intra-articular, intraperitoneal, ophthalmic (e.g., in an ophthalmic preparation such as eye drops, intraocular injections, ointments), aerosol, irrigant, peritoneal lavage, endobronchial and intrathecal administration.

In some embodiments, the other active ingredient is administered topically, orally, intravenously, nasally, ocularly, or transdermally. In some embodiments, the other active ingredient is administered topically. In some embodiments, the other active ingredient is administered orally. In some embodiments, the other active ingredient is administered intravenously. In some embodiments, the other active ingredient is administered nasally. In some embodiments, the other active ingredient is administered ocularly. In some embodiments, the other active ingredient is administered transdermally.

In some embodiments, an amount (e.g., a therapeutically effective amount) of a compound or pharmaceutically acceptable salt thereof is sufficient to increase a rate of clearance of a biofilm in a subject, for example, compared to the same subject were it left untreated. In some embodiments, an amount (e.g., a therapeutically effective amount) of a compound or pharmaceutically acceptable salt thereof is sufficient to increase the rate of clearance of a biofilm by at least about 20%, for example, by at least about: 50%, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold or 100-fold.

In some embodiments, an amount (e.g., a therapeutically effective amount) of a compound or pharmaceutically acceptable salt thereof is sufficient to reduce the quantity of a population of Gardnerella vaginalis in the vagina by at least about 10%, for example, by at least about: 15%, 20%, 25%, 30%, 35%, 40%, 45%, 50%, 55%, 60%, 65%, 70%, 75%, 80%, 85%, 90%, 95% or 99%, or by about: 10-99%, 15-99%, 15-95%, 20-95%, 20-90%, 25-90%, 25-85%, 30-80%, 35-80%, 35-75%, 40-75%, 40-70%, 45-70%, 45-65%, 50-65% or 50-60%. In some embodiments, an amount (e.g., a therapeutically effective amount) of a compound or pharmaceutically acceptable salt thereof is sufficient to reduce the quantity of a population of Gardnerella vaginalis in the vagina by at least about 30%.

### Compositions of the Disclosure

In some embodiments, a monosaccharide is incorporated into a formulation for therapeutic administration (e.g., a pharmaceutical composition). In some embodiments, a composition comprising a glycopolymer of a monosaccharide is incorporated into a formulation for therapeutic administration (e.g., a pharmaceutical composition).

In some embodiments, a pharmaceutical composition further comprises one or more pharmaceutically acceptable carriers or diluents. In some embodiments, a pharmaceutical composition further comprises one or more additional therapeutics, i.e., therapeutic agents (e.g., an antibiotic). Pharmaceutical compositions may be formulated into preparations in, for example, solid, semi-solid, liquid or gaseous forms, such as capsules, gels, granules, microspheres, ointments, powders, solutions, drops, and tablets. Defined, or semi-defined mucin glycan compositions may be formulated for various routes of administration, for example, oral formulations, intravenous formulations, or in the form of a douche. In some embodiments, a defined, or semi-defined mucin glycan composition is formulated into an ointment.

Without wishing to be bound by theory, soluble glycans may be metabolized, but those bonded to a polymer backbone are less likely to be metabolized. Binding the glycans to a surface concentrates the glycan and provides a medium for delivery the glycan to a particular location, namely, at or near the site of infection. Many different surfaces are suitable, including polymers (including bottlebrush polymers and star polymers) and dendrimers. The Exemplification describes experiments with two different types of polymers, a silk fibroin and a norbornene polymer, but a variety of polymers are suitable so long as they are non-toxic when administered as described herein.

### Mucins

Mucins are heavily O-glycosylated glycoproteins that are found in mucous secretions (secreted mucins) and on the cell surface (membrane-bound (transmembrane) mucins). Secreted mucins include gel-forming mucins and non-gel-forming (soluble) mucins.

Mucin genes are expressed in a tissue- and/or region-specific fashion, for example, in the airway, digestive system, reproductive system, and different regions of the gastrointestinal tract. About 20 different mucin genes have been cloned, including gel-forming mucin genes such as MUC2, MUC5AC, MUC5B, MUC6 and MUC19; soluble mucin genes such as MUC7, MUC8, MUC9 and MUC20; and transmembrane mucin genes such as MUC1, MUC3A, MUC3B, MUC4, MUC12, MUC13, MUC15 and MUC21.

Non-limiting examples of mucin genes include human MUC1 (e.g., GenBank: AAA60019.1, UniProtKB/Swiss-Prot: P15941.3, Gene ID 4582), porcine MUC1 (e.g., NCBI: XP_020945387.1), human MUC2 (e.g., GenBank: AAB95295.1, Gene ID 4583), porcine MUC2 (e.g., NCBI: XP_020938243.1), human MUC5AC (e.g., GenBank: ABV02582.1, UniProtKB/Swiss-Prot: P98088.4, Gene ID 4586), porcine MUC5AC (e.g., NCBI: XP_020938242.1), human MUC5B (e.g., UniProtKB/Swiss-Prot: Q9HC84.3, Gene ID 727897), porcine MUC5B (e.g., NCBI: XP_020938146.1), human MUC6 (e.g., GenBank: AZL49144.1) and porcine MUC6 (e.g., NCBI: XP_020938133.1).

Additional non-limiting examples of mucin genes include MUC2 (e.g., HomoloGene 130504, 131905, 132025, or 133451), MUC5AC (e.g., UniGene IDs 3881294, 1370646, 1774723, 1133368, 441382, and 5878683, HomoloGene 130646; Gene ID 100170143; and reference sequences AAC48526, AAD19833, and AAD19832), MUC5B (e.g., HomoloGene 124413), MUC6 (e.g., HomoloGene 18768), MUC19 (bovine submaxillary mucin (BSM), e.g., Gene ID 100140959; HomoloGene 130967; and reference protein sequence XP_0035861 12.1).

### Mucin Glycans

A mucin protein comprises an amino region and/or a carboxy region that are cysteine-rich and a central region enriched in serine and/or threonine residues. Native mucin glycans are typically built upon an N-acetylgalactosamine that is O-linked via its C-1 hydroxyl to serine or threonine residues of a mucin protein. The monosaccharide unit or series of monosaccharide units that, in a native mucin glycan, would be O-linked via the C-1 hydroxyl of the monosaccharide unit or first monosaccharide unit in the series of monosaccharide units, respectively, to a serine or threonine residue of the mucin protein is also referred to herein as the “glycan core” or “mucin glycan core.”

### Exemplification

G. vaginalis is one bacterial species that has been heavily implicated as an initiator of severe bacterial vaginosis. This microbe has two main phenotypic behaviors that contribute to its virulence: its ability to form biofilms that allow it to persistently colonize the vaginal epithelium,²⁹ and the secretion of the cholesterol-dependent cytolysin vaginolysin, which lyses host epithelia, leading to inflammation.^{30,31} At the same time, this organism is found in the vaginas of symptomless patients,^{24,32} suggesting that it is capable of being domesticated in the right conditions. This led us to hypothesize that factors in host mucus may cause G. vaginalis to suppress its virulent behaviors.

Cervical mucus is formed of the mucin MUC5B, a long protein polymer. MUC5B is a glycoprotein, meaning that it is post translationally modified by the addition of sugars, which contribute up to 80% of its molecular weight. Individual mucin samples can contain dozens of glycan structures, built from the same 5 monosaccharide sugars: D-galactose (Gal), N-acetyl D-galactosamine (GalNAc), N-acetyl D-glucosamine (GlcNAc), L-fucose (L-Fuc), and N-acetyl D-neuraminic acid (NeuNAc, or sialic acid). Since it is difficult to get large quantities of cervical mucus for our studies, we instead isolated MUC5B from fresh saliva, which is highly similar to MUC5B expressed in the vaginal tract.³³ We then reconstituted the mucin into gels at higher concentrations, creating a model to study bacterial behavior.

These mucin proteins have many functions in modulating the microbial behavior.³⁴ Canonically, they are a physical barrier, which helps them prevent bacteria from reaching the epithelial surface, and can spatially distribute microbes, creating different niches.³⁵ Secondly, mucin glycans can be a source of diverse and complex nutrients, which can metabolically shape the microbiota.³⁶ Glycans may select for beneficial microbes which produce specific glycosidases, as well as facilitate cooperation across species which produce complementary degradative enzymes. Recently, a third function of mucins has been discovered, which is as an active regulator of microbial virulence. In this model, microbes are able to sense and respond to the glycan structures on healthy mucus.³⁷ Mucin and mucin glycans have non-nutritive effects on several opportunistic pathogens across kingdoms, where the transcriptomes of these microbes change in response to healthy mucin, resulting in decreases of biofilm formation, toxin production, quorum sensing, hyphae formation, and other virulence factors.³⁸⁻⁴⁰ We therefore hypothesize that healthy mucin environments could play a role in preventing pathogen virulence and outgrowth in the vagina.

Here, we studied the influence of healthy vaginal mucin on Gardnerella vaginalis, in order to understand more about how natural host defense mechanisms alter the behavior of opportunistic microbial pathogens, and to leverage this understanding to create interventions that could potentially treat BV without harming commensal Lactobacilli.

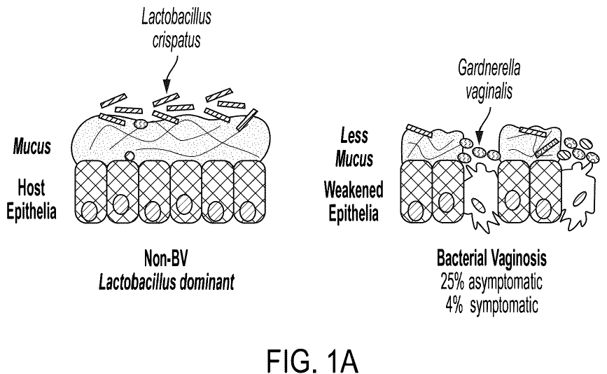

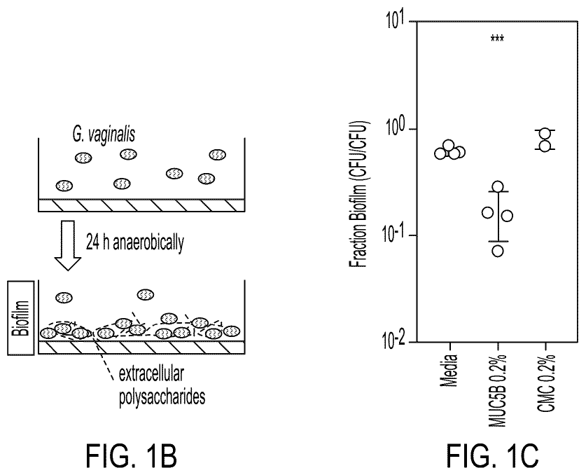

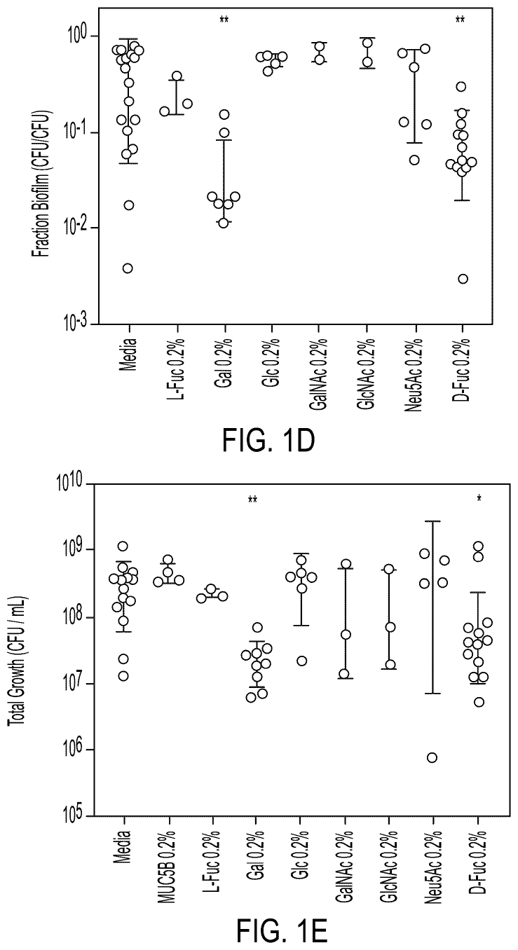

To begin, we cultured G. vaginalis 14018 biofilms in NYCIII media anerobic conditions in the presence or absence of a 0.2 wt % MUC5B mucin gel (FIG. 1B). We find that mucin is able to reduce biofilm formation of G. vaginalis by 76% (FIG. 1C). This effect is somewhat unique to mucin, since supplementation with carboxymethyl cellulose (CMC), a partially synthetic polymer that shares many of the physical properties of mucin gels, did not reduce biofilm formation. It is important to note that MUC5B reduced biofilm formation in proportion to the total bacterial population, that is, MUC5B did not have any detrimental impact on G. vaginalis growth (FIG. 1E). Together, these results suggest that the mucin glycoprotein disrupts bacterial biofilm formation not solely by physical disruption nor by killing the bacteria.

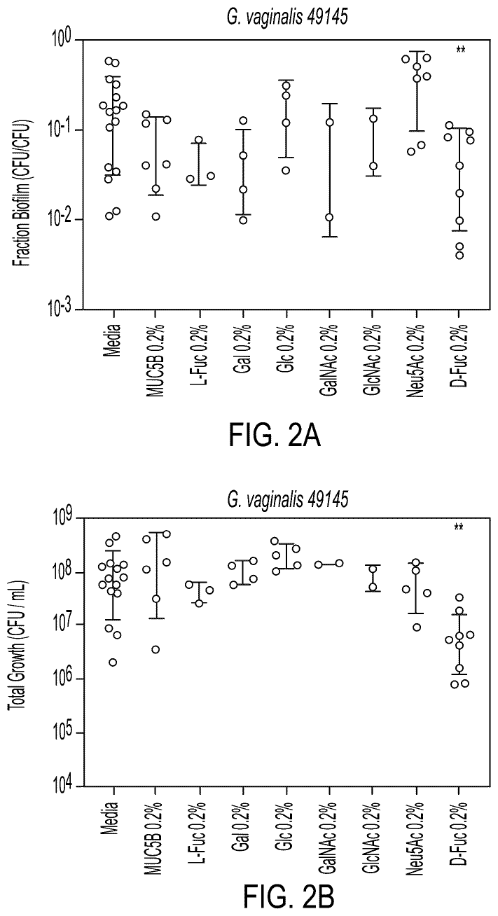

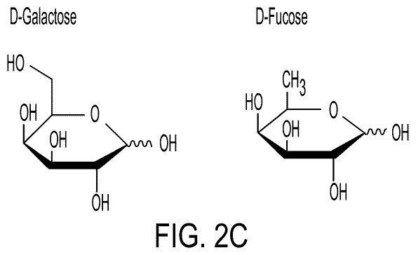

This opens the possibility that mucin's glycans may be responsible for its influence on bacterial behavior. To probe this, we tested whether the monosaccharides that compose MUC5B's complex glycans influenced G. vaginalis biofilm formation (FIG. 1D). We found that of L-Fuc, Gal, GalNAc, GlcNAc, and NeuNAc, only galactose was able to reduce proportional biofilm formation. At 0.2 wt %, galactose supplementation reduced biofilm formation by 85%. Next, we tested 6-deoxy-D-galactose (D-fucose, D-Fuc), which is structurally very similar to galactose (FIG. 2C). At 0.2 wt %, D-fucose was able to reduce biofilm formation by 72%. Interestingly, unlike MUC5B, galactose and D-fucose did suppress growth of G. vaginalis, by 90% and 75% respectively (FIG. 1D) in glucose-rich media. However, galactose and D-fucose did not diminish G. vaginalis 14018 growth in NYCIII media without glucose (data not shown), suggesting that they may be reducing growth by inhibiting efficient utilization of glucose. Together, these results point to galactose as a potent inhibitor of G. vaginalis biofilm formation and growth.

Next, we sought to determine whether these effects were unique to the 14018 strain of G. vaginalis. Thus, we compared against another strain, Gardnerella vaginalis 49145, which was isolated from a patient with bacterial vaginosis. We observed that 49145 showed a similar pattern to 14018, with a reduction of biofilm formation in the presence of MUC5B, galactose, and D-fucose (FIG. 2A). Further, treatment with D-fucose suppressed the growth of 49145 in glucose-rich media (FIG. 2B).

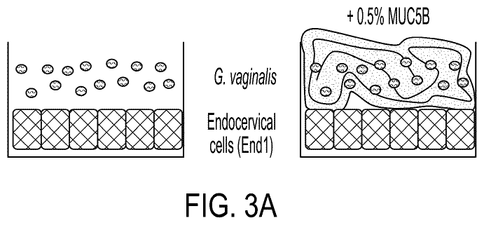

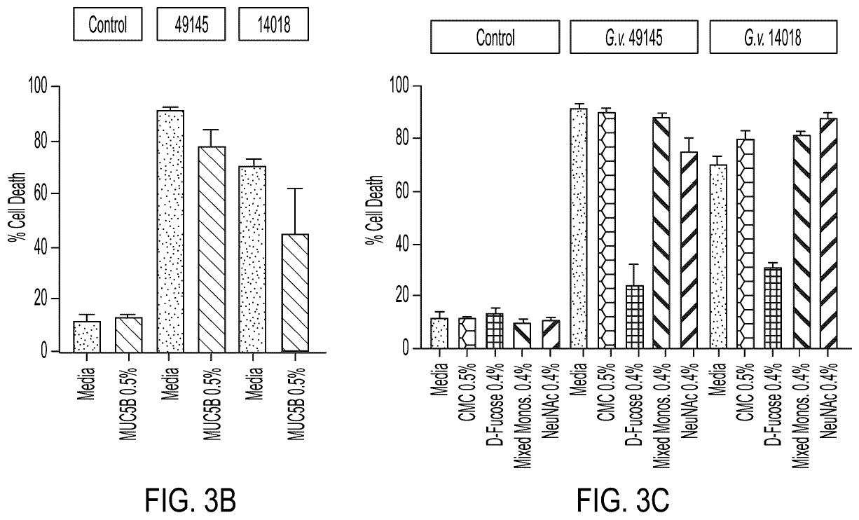

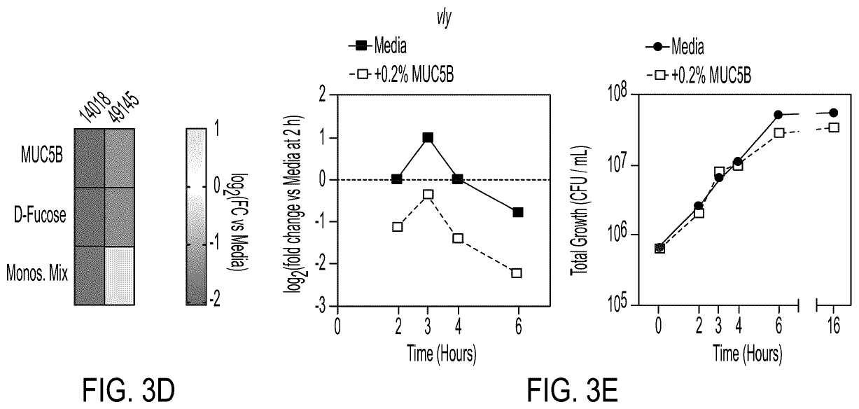

Given that mucin and D-fucose are able to suppress biofilm formation by G. vaginalis 49145 and 14018, we next wondered whether this effect translated into reduced virulence against epithelial cells. To test this, we used an endocervical cell line model (End1), introduced these strains as a pathogen challenge, and then measured the viability of the endocervical cells after 24 hours (FIG. 3A). In the control, challenging End1 cells with either strain of Gardnerella results in almost 90% cell death. However, when mucin is added to these cells, which themselves don't produce gel-forming mucin, we see a significant reduction in cell death (FIG. 3B). In comparison, our polymer control CMC has no effect (FIG. 3C), suggesting that the physical barrier of mucin is not its main protective mechanism. On the other hand, D-fucose was highly effective in reducing endothelial cell killing by G. vaginalis (FIG. 3C). Of note, a pool of mucin monosaccharides did not affect cell killing. Additionally, sialic acid, which has been hypothesized to contribute to G. vaginalis virulence, 41 did not add to or protect against cell killing.

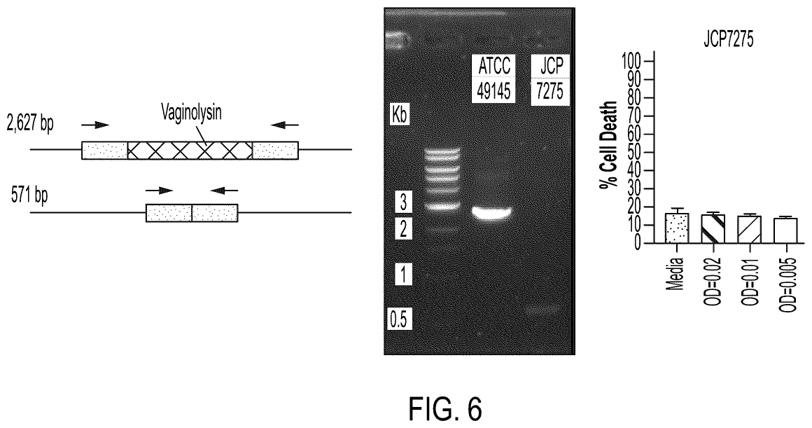

Since the barrier function of mucin does not seem sufficient for its effects, we expect that mucin may be acting as a biochemical signal to reduce virulence. We used RT-qPCR to monitor the expression of vaginolysin (vly), a cholesterol-dependent cytolysin that kills epithelial cells and is a major contributor to Gardnerella virulence (FIG. 3D). We find that MUC5B and D-fucose are able to significantly reduce vly expression in 14018 and 49145, suggesting that they are affecting the End1-cell killing phenotype by altering the transcriptional response of these microbes. Further, vly expression was suppressed by MUC5B throughout exponential growth. This suggests that healthy MUC5B helps protect the vaginal epithelia by preventing pathogens like G. vaginalis from expressing toxins. This mechanism was supported by evidence that strains (such as JCP7275) that naturally lack the gene for vaginolysin are not pathogenic to the End1 cell line (FIG. 6).

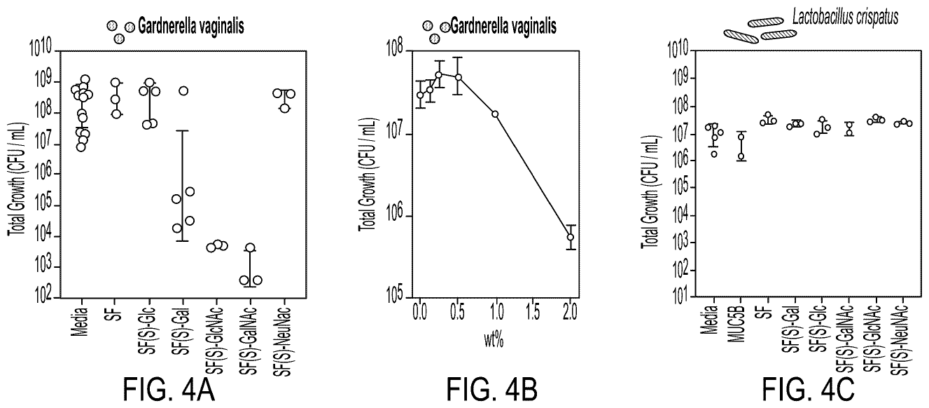

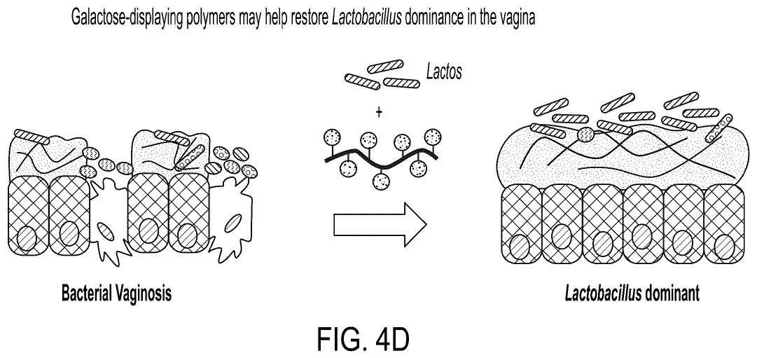

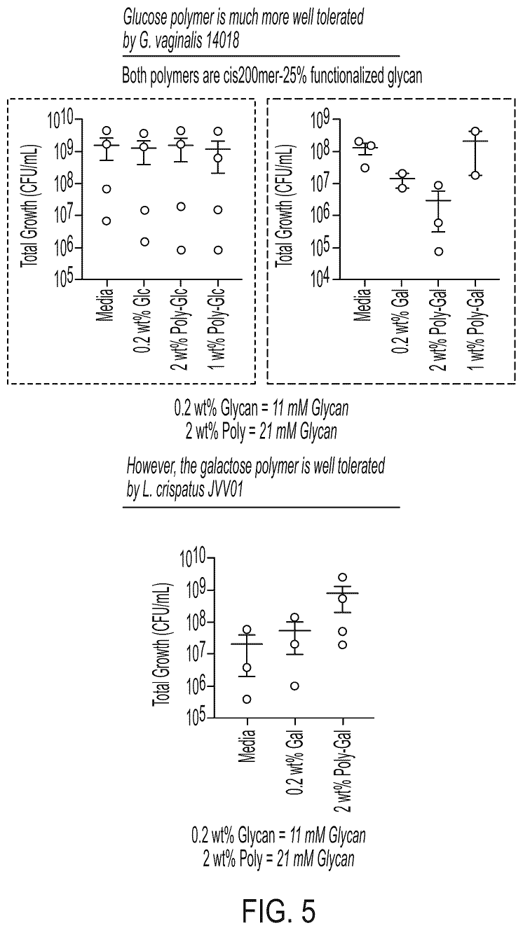

We next sought to determine if grafting galactose onto a polymer backbone could enhance its effects. While 0.2 wt % of soluble galactose inhibited growth in glucose rich media by 90%, when galactose was tethered onto silk fibroin, treatment with 2.5 wt % SF(S)-Gal reduced growth by 99.7%, an improvement of 2-3 orders of magnitude (FIG. 4A). Since the polymers are about 10-20% sugar by weight, the bacteria were exposed to equivalent quantities of galactose. Therefore, it seems that some effect of having the sugar grafted to a polymer backbone greatly increases its efficacy. Additionally, it appears that this effect is agnostic to polymer backbone, as galactose brush polymers with a norbornene backbone 42 produced an almost identical effect at similar treatment strengths and functionalization densities (FIG. 4B). Importantly, this effect was not agnostic to which sugar was displayed, as glucose grafted polymers did not impact growth of G. vaginalis 14018 (FIG. 4A). Additionally, the silk fibroin backbone control did not alter the bacteria in any way. Interestingly, it does appear that SF(S)-GalNAc and SF(S)-GlcNAc also prove toxic to G. vaginalis, although the soluble monosaccharides GalNAc and GlcNAc have no effect.

Finally, we tested whether the silk glycopolymers impacted the growth of a commensal bacterium Lactobacillus crispatus, which is a lead candidate for most vaginal probiotics. Here, we observed that none of the silk glycopolymers had a detrimental effect on the growth of strain JV001 (FIG. 4C). The lack of impact on this strain suggests that such mucin-inspired glycopolymers could be used to modulate the growth dynamics of communities to promote the survival of beneficial organisms (FIG. 4D).

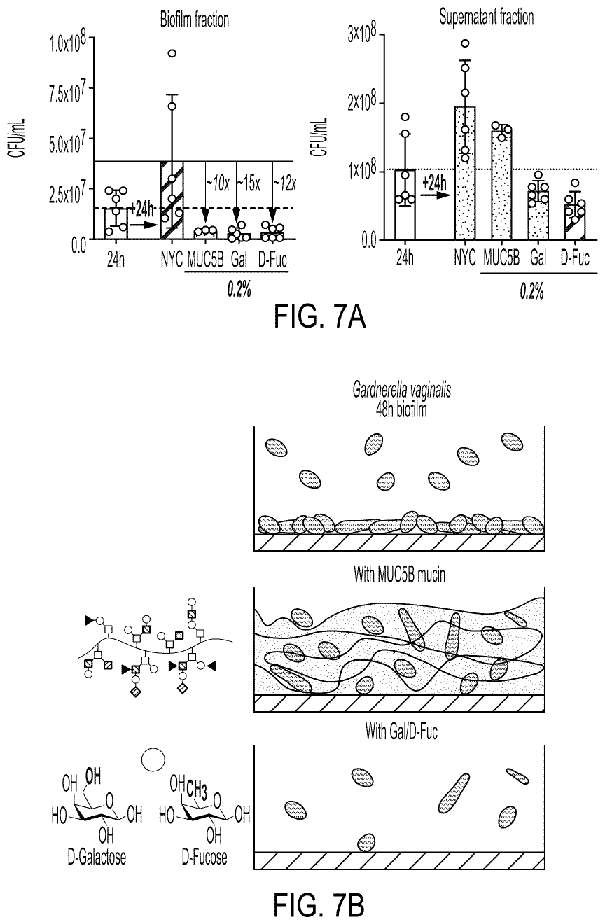

We discovered that MUC5B mucin, its associated monosaccharide galactose (=D-Gal) and an unnatural analogue D-fucose (=D-Fuc) are able to robustly dissolve pre-established Gardnerella vaginalis biofilms. The observed reduction constitutes a 10-15-fold decrease in the number of colony-forming units in the biofilm fraction (FIG. 7A). For MUC5B mucin condition reduction in the biofilm fraction leads to increase in the cell counts in the supernatant above the biofilm, indicating that the primary mechanism of dispersion is a shift from biofilm state to planktonic state without the loss of viability. In contrast, D-Gal and D-Fuc conditions lead to the reduction in viable cells in both biofilm and supernatant fractions, suggesting that killing takes place (FIG. 7B).

We hypothesized that the biofilm disruption activity of mucin glycan Gal and its analogue D-Fuc has the potential to increase the susceptibility of Gardnerella vaginalis to antibiotic metronidazole. For this we performed a minimum biofilm eradication assay wherein established biofilms were exposed to metronidazole alone, glycans alone or the combination thereof. Across several metronidazole concentrations we observe a synergistic effect between the antibiotic and monosaccharides, leading to a significant increase in the magnitude of the biofilm disruption achieved in the combined conditions.

### Discussion

The vaginal canal is coated with the mucin MUC5B and is colonized by a variety of commensal Lactobacilli. Here, we targeted the Gardnerella vaginalis, an opportunistic pathogen that can contribute to symptomatic bacterial vaginosis, but is carried asymptomatically in some patients. We hypothesized that mucin may alter pathogenic behaviors of G. vaginalis, and indeed, exposure to MUC5B limited its biofilm formation, toxin production, and killing of endocervical cells. Next, we identified that galactose and a structurally similar monosaccharide, D-fucose, were able to reproduce mucin's virulence suppression. Additionally, these molecules limited G. vaginalis growth in glucose-rich media, suggesting that they may function by rerouting microbial metabolism. Finally, we saw that when galactose was grafted onto silk fibroin polymers (SF-Gal), it was extremely toxic to G. vaginalis, but not L. crispatus. Additionally the Galactose tethered to a norbornene backbone had a similar effect, as did SF-GalNAc and SF-GlcNAc. Together, these results suggest that mucin and galactose play an important role in maintaining health in the vaginal microbiome.

Here, we showed that mucin, galactose, and the galactose-similar molecule D-fucose are able to inhibit biofilm formation by the vaginal pathogen G. vaginalis. Further, galactose suppresses bacteria growth by one log in glucose-rich media, an effect that is amplified by several orders of magnitude when galactose is grafted onto a polymer backbone. The mechanism by which these antimicrobial polymers function is still unclear, but we hypothesize that they interfere with the efficient transport or utilization of nutrients, or they disrupt membrane integrity.

This study demonstrated that both natural mucin and synthetic glycopolymers have a profound effect on bacterial virulence. This illustrates the importance of mucin as a natural protective mechanism the host uses to disrupt bacterial virulence. On the other hand, the galactose-based synthetic polymers appear as a novel method for inhibiting bacterial growth. Most antimicrobial polymers are highly positively charged, or zwitterionic, and act on broad categories of bacteria by disrupting their naturally negatively charged membranes.⁵¹ These polymers appear to act by an entirely orthogonal mechanism that enables them to be selective, as demonstrated by their impotence against Lactobacillus crispatus. These materials provide a case study for the development of mucin-inspired antimicrobial agents that can selectively target pathogenic organisms while leaving beneficial commensals intact. Such therapies could provide a new paradigm of treating infections, complementing vaginal microbiome transplants by aiding in the reduction of pathogenic bacteria when probiotic strains are introduced.

Here, we explored one method of creating mucin-inspired glycan-bearing materials: a bottlebrush structure on a polymer backbone. However, we expect that there is significant space for exploring alternative architectures, such as surfaces or thicker gels. We observed that grafting the same sugar onto either silk fibroin or norbornene resulted in a bioactive polymer, indicating that the specific backbone identity is not necessarily essential for function; rather, it seems that the act of being grafted increases sugar activity. Additionally, materials which blend different bioactive sugar monomers could be created, enabling the simultaneous targeting of multiple microbial phenotypes.

With the rise of clinical antimicrobial resistance, these materials, which treat infections by hijacking nutrient sensing pathways, rather than directly killing bacteria, present a novel strategy for treating infections that can target pathogens while leaving the commensal microbiome intact.

### Materials and Methods

### Bacterial Strains and Reagents

The bacterial strains Gardnerella vaginalis ATCC 14018 and ATCC 49145 were obtained from ATCC and were originally isolated from people with active bacterial vaginosis. JCP7275 was obtained from BEI Resources. Bacteria were cultivated in modified ATCC NYCIII media or brain-heart infusion (BHI; BD 237500) 1.5% agar plates at 37° C. in airtight containers with Mitsubishi AnaeroPack-Anaero Anaerobic Gas Generators (Thermo Scientific; R681001), which consume oxygen and produce high concentrations of carbon dioxide. NYCIII media was prepared in 2× concentrations, flash cooled, and thawed in an anaerobic chamber the day before use. Briefly, modified NYCIII media was prepared in 250 ml batches in two steps. First, 2 g HEPES, 7.5 g Proteose Peptone No. 3 (BD 211693), 2.5 g NaCl, and 177.5 ml miliQ-distilled water were mixed, adjusted to pH 7.3, and autoclaved for 15 minutes. When the solution had cooled to room temperature, we added 12.5 ml of yeast extract solution (prepared by dissolving wt % Bacto Yeast Extract in miliQ-distilled water and sterilizing with a 0.2 μm syringe filter), ml of heat-inactivated horse serum (Millipore Sigma; H1138), 10 ml of 50 wt % 0.2 μm sterile filtered glucose solution. Media was then aliquoted into 10 ml tubes, flash cooled in liquid nitrogen, and stored at −80° C.

Mucin Purification from Pooled Saliva

Submandibular saliva was collected from informed consenting volunteers as described previously.⁵³ Saliva donations from five donors were pooled before MUC5B was purified using liquid chromatography.⁴⁰ Purified MUC5B was flash cooled, lyophilized, and stored at −80° C. Before use, MUC5B was rehydrated in Milli-Q-purified water and agitated at 4° C. overnight. Protocols involving samples from human participants were approved by the Massachusetts Institute of Technology's Committee on the Use of Humans as Experimental Subjects.

### Biofilm and Growth Assays

Overnight cultures of G. vaginalis were inoculated from glycerol stocks and grown for 12-24 hours in NYCIII media at 37° C. in an anaerobic chamber. Biofilms were prepared by diluting overnight cultures 1:20 into 50-100 μl of NYCIII media and any additives in a 96-well polystyrene plate for a starting inoculum of OD₆₀₀˜0.1 or 10⁵-10⁶ CFU per ml. The cultures were then incubated statically for 24 hours at 37° C. in an anaerobic chamber.

To measure total growth and relative biofilm formation, the number of cells in the biofilm and in suspension were counted using a colony forming unit (CFU) assay as previously described. 7 Briefly, the supernatant was removed and transferred to a new 96-well plate, the biofilm was washed twice with 100 μl of phosphate-buffered saline (PBS), and the washes were added to the supernatant. The biofilm was then detached and resuspended in 100 μl of PBS by vigorously scraping with a pipette tip for 30 s. Each culture fraction was then serially diluted and plated on BHI agar. Colonies were counted after 48 hours of growth at 37° C. in an anaerobic chamber. The fraction biofilm was calculated as (biofilm CFU per ml)/(biofilm CFU per ml+supernatant CFU per ml). The total growth was calculated as biofilm CFU per ml+supernatant CFU per ml. All of the conditions had at least three biological replicates (individual data points shown on graphs).

### Gene Expression Analysis

A table of the primers used in this study is provided in Table 1 (SEQ ID NOS: 1-4). Gene expression analysis was performed using quantitative PCR with reverse transcription (RT-qPCR) as previously described. 40 Briefly, subcultures were grown as described above, but in PCR tubes. After 3 hours of growth, cells were centrifuged, the supernatant was removed, and the pellet was flash cooled in liquid nitrogen. Total nucleic acids were then extracted using the MasterPure Complete RNA Purification kit (Lucigen). Genomic DNA was removed using the Turbo DNA-free kit (Ambion). Total RNA was measured using an Agilent 2100 Bioanalyzer (Agilent Technologies) and stored at −80° C. cDNA was synthesized with the Protoscript II First-Strand cDNA Synthesis kit (New England Biolabs). About 2 ng of cDNA was used as a template for qPCR using SYBR Green Master Mix (Thermo Fisher Scientific) performed using the Cycler 480 II Real-time PCR Machine (Roche). Gene expression changes were calculated as the mean change in qPCR cycle threshold compared to 16S rRNA (ΔCₜ) and are reported as log₂[fold change]=ΔCₜ_media−ΔCₜ_sample. Each sample was analyzed with at least three technical replicates.

### Identification of Vly-Strains

To identify Gardnerella strains lacking vaginolysin (vly), the vly sequence was blasted to the vaginal genomes of Gardnerella strains available through BEI. Strains that did not yield a blast result were grown for microbial gDNA extraction (Qiagen) and tested against the G. vaginalis ATCC49145 strain encoding vly. The presence of vly was determined by PCR amplification of the flanking regions using primers obtained from Cerca 2015. The PCR products were analyzed by gel electrophoresis and sequences by Genewiz to confirm the sequence identity.

### Synthesis of Norbornene-Galactose Polymers

The norbornene polymers were synthesized and characterized as described in Kruger et. al. 2021.⁴² The experiments described utilized cis polymers of 200 units in length and 20% galactose grafting density. Experiments used polymers at 2 wt %.

The overall synthesis of the galactose norbornene polymers was completed as illustrated in Scheme 1. The synthesis of the requisite reactants was completed as follows.

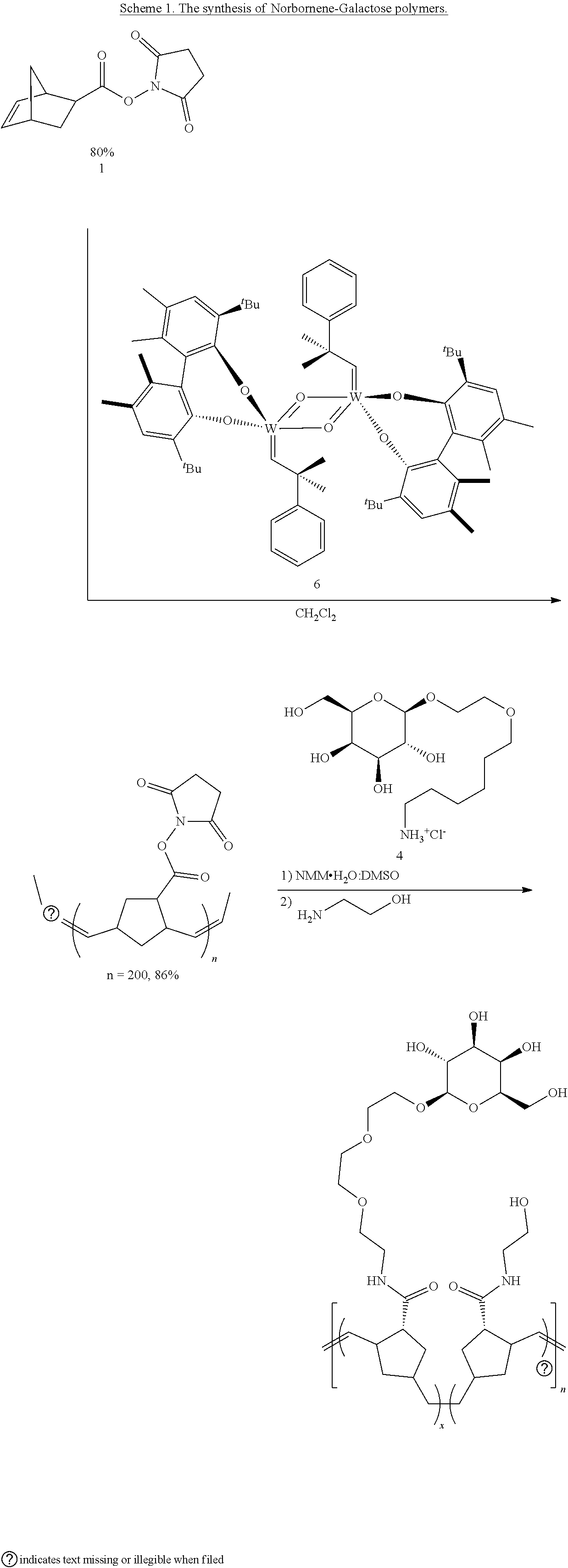

Known compound 4 was synthesized according to a modified procedure for compound 37 in Percec et. al. J. Am. Chem. Soc. 2013. To a 500 mL round bottom flask equipped with a stir bar 5.06 g of protected galactoside S3 (7.21 mmol, 1 equiv.) was added. In a 24 mL vial a solution of 1 M NaOMe in MeOH was freshly prepared by addition of 230 mg sodium metal into 10 mL MeOH. This solution was used to basify at least 140 mL MeOH to pH 9-10 by pH paper. The protected galactoside S3 was dissolved in 130 mL of the pH 9-10 NaOMe/MeOH solution and 1.42 mL piperidine (14.4 mmol, 2 equiv.) was added. The solution was heated to 40° C. and stirred for 48 hours after which the reactant solution was a light champagne color. The reactant solution was concentrated and partitioned between 40 mL each H₂O and EtOAc, and washed with a further 2×40 mL portions EtOAc. The aqueous phase was basified to pH 11 by pH paper with 1-3 g Amberlyst A26 hydroxide form resin and washed with 3×40 mL portions methylene chloride (note that the Amberlyst resin needed to be physically present within the separatory funnel to aid in piperidine removal). The aqueous phase was transferred to a 500 mL round bottom flask and the residual methylene chloride was removed under reduced pressure. The aqueous phase was passed over a 0.22 μm syringe filter, neutralized by pH paper with 1-10 mL 1 M HCl, and lyophilized. The product was obtained as a light yellow resinous crystalline solid. The product was generally obtained with residual piperidine contaminant and was extremely hygroscopic. Consequently, the mass of product generally exceeded that of a theoretical 100% yield, however, the material was still readily grafted in the desired stoichiometry to polymers without accounting for these impurities. No Rf was obtained since the product would not migrate by normal phase TLC in any solvent system tested and streaked so heavily on reverse phase TLC as to be impractical. Average Yield: ≥99%. Minimum Yield: 97%. Maximum Yield: ≥99%. 1H NMR (600 MHz, deuterium oxide) δ 4.35 (d, J=7.9 Hz, 1H), 4.02 (dt, J=11.4, 4.0 Hz, 1H), 3.85 (d, J=3.5 Hz, 1H), 3.80-3.74 (m, 1H), 3.74-3.64 (m, 12H), 3.62 (dd, J=7.8, 4.3 Hz, 1H), 3.58 (dd, J=9.9, 3.4 Hz, 1H), 3.46 (abq, J=9.9, 7.9 Hz, 1H).

Compound 1 was synthesized according to a modified procedure for compound 15-NHS in Werther, et al. Chem.—A Eur. J. 2017. To a flame dried N₂ flushed 100 mL round bottom flask 2.0 g exo-norbornene-5-carboxylic acid S5 (14.5 mmol, 1 equiv.), 2.33 g N-hydroxysuccinimide (20.2 mmol, 1.4 equiv.), 3.05 g 1-ethyl-3-(3-dimethylaminopropyl)carbodiimide·HCl (15.9 mmol, 1.1 equiv.) were all added and dissolved in 24.1 mL dry methylene chloride. The reaction was allowed to proceed overnight before concentrating. The reactant solution remained clear and colorless throughout the reaction. The concentrate was partitioned between 90 mL each EtOAc and H₂O, and the aqueous phase was extracted with a further 2×90 mL portions EtOAc. The organic phases were combined and washed with 2×45 mL portions saturated NH₄Cl, 2×90 mL portions saturated NaHCO₃, 1×90 mL portion brine, dried over MgSO₄, and concentrated. Product was obtained as an off-white solid with R_{f}=0.48 on 30% EtOAc:Hexanes. Average Yield: 80%. Minimum Yield: 73%. Maximum Yield: 88%. ¹H NMR (600 MHz, chloroform-d) δ 6.23 (dd, J=5.7, 3.0 Hz, 1H), 6.17 (dd, J=5.7, 3.1 Hz, 1H), 3.32-3.24 (m, 1H), 3.02 (m, 1H), 2.86 (d, J=6.6 Hz, 5H), 2.53 (ddd, J=9.0, 4.5, 1.4 Hz, 1H), 2.07 (dt, J=12.0, 3.8 Hz, 1H).

The norbornene-galactose polymer (“Cis 200mer”) was synthesized according to the modified procedure for entry 8 (main text) or entry 9 (SI) in Yan et al. Organometallics 2019. All procedures for carrying out these cis-polymerization were performed in a dry N2 filled glovebox. To a 24 mL vial equipped with a stir bar, 250 mg NHS-monomer 1 (1.06 mmol, 150 equiv.) was added and dissolved in 3-5 mL dry methylene chloride. In a separate 24 mL vial, a solution of 9.7 mg (7.1 μmol, 1 equiv.) catalyst 6 in 2-4 mL dry methylene chloride was prepared. The catalyst formed a bright orange-yellow solution. Note that catalyst 6 typically yielded polymers of greater DP than targeted. To compensate for this, low DP reactions were set up using lower equivalents of monomer to catalyst than theoretically required to yield a polymer of a particular length. Here, a ratio of monomer to catalyst of 150:1 was found to yield approximately DP=200 by 1H NMR end-group analysis. The catalyst solution was added rapidly to the monomer solution and the reaction was allowed to proceed for 30 minutes before removing from the glovebox. The reactant solution rapidly took on a burnt-orange color after initiation. The reactant solution was precipitated dropwise into 40 mL MeOH, centrifuged, and decanted. The product was obtained as a white rock-like solid from precipitation. No Rf was measured for the polymers. DP=200 by 1H NMR end-group analysis where the phenyl signal at 7.44-7.13 ppm was integrated for 5H and the integral of the signal at 1.82 ppm was taken to determine DP. Ð=2.06. Average Yield: 86%. Minimum Yield: 86%. Maximum Yield: 86%. 1H NMR (600 MHz, methylene chloride-d2) δ 7.44-7.13 (m, 5H) 5.58-5.12 (brm, 731H, overlap with solvent), 3.45 (brm, 663H), 2.81 (brm, 1100H), 2.42-1.99 (brm, 440H), 1.82 (brm, 227H), 1.70-1.32 (brm, 163H), 1.25 (brm, 239H).

### Synthesis of Silk Polymers

The silk fibroin polymers were synthesized and characterized as described below. The experiments utilized polymers where glycans were grafted onto the serine residues of boiled silk fibroin. Experiments used polymers at 2.5 wt %.

### Extraction of Aqueous Silk Solution

Silk fibroin (SF) solutions were extracted using our prior protocols. 54 Briefly, 5 grams of Bombyx mori silkworm (Tajima Shoji Co. Ltd., Yokohama, Japan) cut cocoons were degummed in 2 L of 0.02 M Na 2 CO 3 solution (Sigma-Aldrich, St. Louis, MO) in a glass beaker for 60 minutes to remove the sericin protein coating. Degummed silk was collected and rinsed with deionized water (DI) in 4 L (20 minutes, 3 times), followed by drying at room temperature in a fume hood overnight. The dried degummed SF fibers were solubilized in 9.3 M Lithium Bromide (LiBr) (Sigma-Aldrich, St. Louis, MO) solution, in a preheated oven at 60° C. for 4 h. After 4 h, light brown color SF solution was obtained which was dialyzed against 4 L of DI water with six water changes for 48 h (water changes at 1, 2, 4, 24, 36, and 48 h). The dialysis was performed with dialysis tubing (3,500 MWCO, Thermo Scientific, Rockford, IL). After 48 h, the dialyzed silk solution was centrifuged (9,000 RPM, 20 min, 4° C., 2 times) to remove insoluble aggregates. The concentration of the regenerated SF solution was calculated by drying a known mass of the aqueous SF solution in a weigh boat in an oven at 60° C. overnight and assessing the mass of the remaining solid film. The aqueous SF solution was stored at 4° C. until further use.

### Synthesis of SF(S)—COOH

Aqueous SF solution was carboxylated by nucleophilic substitution reaction in a highly alkaline reaction environment, in the presence of chloroacetic acid (Sigma-Aldrich, St. Louis, MO) at pH ˜13.5. Briefly, the pH of the 1M chloroacetic acid was adjusted by adding freshly prepared 10M sodium hydroxide (NaOH) solution to raise the pH to 13.3-13.5. At pH 13.5, reconstituted SF solution (0.6 wt %) was added dropwise to the mixture. Addition of SF solutions can decrease the pH which was further adjusted to pH 13.5 by dropwise addition of 10 M NaOH solution. The solution was stirred gently for 1 h at RT. After 1 h, sodium phosphate monobasic (NaH 2 PO 4) (Sigma-Aldrich, St. Louis, MO; Lot #015K0024, 4 mg/mL) was added to the reaction mixture and stirred. Addition of NaH 2 PO 4 decreased the pH of the mixture. The pH of the solution was adjusted to 7-7.5 by slow dropwise addition of 10M hydrochloric acid (HCl) (Sigma-Aldrich, St. Louis, MO) solution. At pH 7-7.5, the reaction mixture was stirred for 30 minutes at RT. After 30 minutes, the carboxylated SF solution was dialyzed against DI water for 72 h with six water changes (1 h, 2 h, 4 h, 24 h, 48 h, and 72 h) to remove byproducts and impurities. The dialysis was performed with dialysis tubing (3,500 MWCO, Thermo Scientific, Rockford, IL) in 4 L with gentle stirring. After dialysis, the carboxy-modified SF solutions were filtered in a sterile cell strainer with 40 μm mesh size (Thermo Scientific, Rockford, IL). After filtration, the solutions were frozen at −80° C. overnight followed by lyophilizing for at least 72-96 h. The lyophilized powders were collected and stored at 4° C. until further use.

### Synthesis of SF(S)-EDA

The carboxylated SF (SF(S)—COOH) was covalently conjugated with primary amines of ethylene diamine (EDA) hydrochloride (Sigma-Aldrich, St. Louis, MO) by carbodiimide coupling in the presence of N-3-Dimethyl amino propyl-N′-ethyl carbodiimide (EDC) hydrochloride, and N-Hydroxy Succinimide (NHS) (Sigma-Aldrich, St. Louis, MO). Briefly, 2 wt % of the SF(S)—COOH solution was dissolved in 0.1M MES (2-(N-morpholino) ethanesulfonic acid) buffer at pH 6. EDA (10×excess) was weighed and pre-dissolved in ultrapure distilled water (Thermo-Fisher Scientific, Waltham, MA) and added to the SF(S)—COOH solution. The pH was readjusted to 6 by dropwise addition of freshly prepared 1M sodium hydroxide (NaOH) solution. EDC (10×) and NHS (10×) (pre-dissolved in MES buffer, pH 6) were added to the reaction mixture at pH 6. The final MES buffer concentration of the reaction mixture was adjusted to 0.05M by addition of ultrapure distilled water. The reaction was stirred gently at RT for 18 h. After the reaction, the aggregates were filtered in a sterile cell strainer with 40 μm mesh size (Thermo Scientific, Rockford, IL) and dialyzed against DI water for at least 72-96 h with six water changes (1, 2, 4, 24, 48, and 72 h). Dialysis was performed with dialysis tubing (3500 MWCO, Thermo Scientific, Rockford, IL). After dialysis, the solutions were frozen at −80° C. overnight followed by lyophilizing for 72 h. The lyophilized powders were stored at 4° C. until further use.

### Synthesis of SF(S)-GalNAc, SF(S)-GlcNAc, and SF(S)—NeuNAc

The aminated SF solution (SF(S)-EDA) were conjugated with carboxylic acid moieties of 4-carboxybutyl N-acetyl-β-D-galactosaminide ((3-GalNAc-Bu-COOH) (Sussex Research, Ottawa, Canada), 4-carboxybutyl N-acetyl-β-D-glucosaminide (β-G1cNAc-Bu-COOH) (Sussex Research, Ottawa, Canada), or N-Acetylneuraminic acid hydrate (TCI America, Portland, OR) by carbodiimide coupling in the presence of EDC and NHS (Sigma-Aldrich, St. Louis, MO). Briefly, 2 wt % of the SF(S)-EDA was dissolved in 0.1M MES (2-(N-morpholino) ethanesulfonic acid) buffer at pH 6. β-GalNAc-Bu-COOH (3× times molar excess), β-G1cNAc-Bu-COOH (3× molar excess), or N-Acetylneuraminic acid hydrate (2× molar excess) was weighed and pre-dissolved in 0.1M MES buffer and the pH was readjusted to 6 by dropwise addition of freshly prepared 1M NaOH solution. EDC (3×) and NHS (3×) were added to the sugar solution at pH 6 to activate the carboxylic acid. The reaction was readjusted to pH 6 and stirred for 30 minutes at RT. After 30 minutes, SF(S)-EDA solution was dropwise added to the activated solution. The final MES buffer concentration of the reaction mixture was adjusted to 0.05M by addition of ultrapure water. The pH was readjusted to 6 after addition of SF(S)-EDA solution. The reaction was stirred at RT for 18 h. After 18 h, aggregates were filtered through a sterile cell strainer with 40 μm mesh size (Thermo Scientific, Rockford, IL) and dialyzed against DI water for at least 72 h with six water changes (1 h, 2 h, 4 h, 24 h, 48 h, and 72 h). The dialysis was performed with dialysis tubing (3,500 MWCO, Thermo Scientific, Rockford, IL). After dialysis, the solutions were frozen at −80° C. overnight followed by lyophilizing for 72 h. The lyophilized powders were stored at 4° C. until further use.

### Synthesis of SF(S)-GlcN and SF(S)-GalN

SF(S)—COOH was conjugated with amines of D (+)-Glucosamine Hydrochloride (Sigma-Aldrich, St. Louis, MO) by carbodiimide coupling in the presence of EDC and NHS (Sigma-Aldrich, St. Louis, MO) or D (+)-Galactosamine Hydrochloride (Sigma-Aldrich, St. Louis, MO). A 2 wt % of the SF(S)—COOH solution was dissolved in 0.1M MES (2-(N-morpholino) ethanesulfonic acid) buffer at pH 6. D (+)-Glucosamine Hydrochloride (3×) or D(+)-Galactosamine Hydrochloride (3×) was weighed and pre-dissolved in ultrapure distilled water (Thermo-Fisher Scientific, Waltham, MA) and added to the SF(S)—COOH solution. The pH was readjusted to 6 by dropwise addition of freshly prepared 1M sodium hydroxide (NaOH) solution. EDC (3×) and NHS (3×) (pre-dissolved in MES buffer, pH 6) were added to the reaction mixture at pH 6. The final MES buffer concentration of the reaction mixture was adjusted to 0.05M by addition of ultrapure distilled water. The reaction was allowed to stir gently at RT for 18 h. The aggregates were filtered, after 18 h, through a sterile cell strainer (40 μm mesh size) (Thermo Scientific, Rockford, IL) and dialyzed against DI water for at least 72 hours with six water changes (1, 2, 4, 24, 48, and 72 h). Dialysis was performed with dialysis tubing (3500 MWCO, Thermo Scientific, Rockford, IL). After dialysis, the solutions were frozen at −80° C. overnight followed by lyophilizing for 72 h. The lyophilized powders were stored at 4° C. until further use.

### Synthesis of SF (D, E)-EDA

The regenerated silk fibroin was covalently conjugated with primary amines of ethylene diamine (EDA) hydrochloride (Sigma-Aldrich, St. Louis, MO) by carbodiimide coupling in the presence of EDC, and NHS (Sigma-Aldrich, St. Louis, MO). Briefly, 2 wt % of the SF solution was dissolved in 0.1M MES (2-(N-morpholino) ethanesulfonic acid) buffer at pH 6. EDA (10×) was weighed and pre-dissolved in Ultrapure distilled water (Thermo-Fisher Scientific, Waltham, MA) and added to the aqueous SF solution. The pH was readjusted to 6 by dropwise addition of freshly prepared 1M sodium hydroxide (NaOH) solution. EDC (10×) and NHS (10×) (pre-dissolved in MES buffer, pH 6) were added to the reaction mixture at pH 6. The final MES buffer concentration of the reaction mixture was adjusted to 0.05M by addition of ultrapure distilled water. The reaction was stirred gently at RT for 18 h. After the reaction was completed, aggregates were filtered through a cell strainer (mesh size 40 μm) (Thermo Scientific, Rockford, IL) and dialyzed against DI water for at least 72 hours with six water changes (1, 2, 4, 24, 48, and 72 h). Dialysis was performed with dialysis tubing (3500 MWCO, Thermo Scientific, Rockford, IL). After dialysis, the solutions were frozen at −80° C. overnight followed by lyophilizing for 72 h. The lyophilized powders were stored at 4° C. until further use.

### Synthesis of SF (D, E)-GalNAc

The aminated SF solution (SF (D, E)-EDA) were conjugated with carboxylic acid moieties of 4-carboxybutyl N-acetyl-β-D-galactosaminide (β-GalNAc-Bu-COOH) (Sussex Research, Ottawa, Canada) by carbodiimide coupling in presence of EDC and NHS (Sigma-Aldrich, St. Louis, MO). Briefly, 2 wt % of the SF (D, E)-EDA was dissolved in 0.1M MES (2-(N-morpholino) ethanesulfonic acid) buffer at pH 6. β-GalNAc-Bu-COOH (3×times molar excess) was weighed and pre-dissolved in 0.1M MES buffer and pH was readjusted to 6 by dropwise addition of freshly prepared 1M NaOH solution. EDC (3×) and NHS (3×) were added to β-GalNAc-Bu-COOH at pH 6 to activate the carboxylic acid. The reaction was readjusted to pH 6 and stirred for 30 minutes at RT. After 30 minutes, the SF (D, E)-EDA solution was added dropwise to the activated solution. The final MES buffer concentration of the reaction mixture was adjusted to 0.05M by addition of ultrapure water. The pH was readjusted to 6 after addition of SF (D, E)-EDA solution. The reaction was stirred at RT for 18 h. After the reaction was complete, aggregates were filtered through a sterile cell strainer with 40 μm mesh size (Thermo Scientific, Rockford, IL) and dialyzed against DI water for at least 72 h with six water changes (1 h, 2 h, 4 h, 24 h, 48 h, and 72 h). Dialysis was performed with dialysis tubing (3,500 MWCO, Thermo Scientific, Rockford, IL). After dialysis, the solutions were frozen at −80° C. overnight followed by lyophilizing for 72 h. The lyophilized powders were stored at 4° C. until further use.

MUC5B Mucin Purification from Pooled Saliva

Submandibular saliva was collected from informed consenting volunteers as described previously.⁴⁰ Saliva donations from five donors were pooled before MUC5B was purified using liquid chromatography as described previously.⁵⁵ Purified MUC5B was flash cooled, lyophilized, and stored at −80° C. Before use, MUC5B was rehydrated in Milli-Q-purified water and agitated at 4° C. overnight. Protocols involving samples from human participants were approved by the Massachusetts Institute of Technology's Committee on the Use of Humans as Experimental Subjects.

TABLE 1
Primers used in RT-qPCR.

| Gene | FWD | REV | Source |
|---|---|---|---|
| 16S rRNA | SEQ ID NO: 1 | SEQ ID NO: 2 | Salinas et. al. 2018⁵² |
| vly (set 1) | SEQ ID NO: 3 | SEQ ID NO: 4 | Castro et. al. 2015³² |

### MUC5B Mucin and its Specific Monosaccharides Disrupt Established Biofilms of Gardnerella Vaginalis (FIGS. 7A-B)

Gardnerella vaginalis 14018 ATCC strain was obtained from ATCC. Salivary MUC5B mucin was isolated and purified as described previously. Galactose and D-Fucose were obtained from Sigma-Aldrich. AnaeroPacks were purchased from VWR. Prior to the experiment, MUC5B mucin was weighed out and solubilized to desired concentration on a shaker overnight.

To establish mature biofilms, an overnight preculture of Gardnerella vaginalis ATCC 14018 was diluted 1:20 into fresh NYC III medium with 1% glucose and incubated at 37° C. in the anaerobic chamber for 24 h.

Before adding fresh solutions, a set of triplicate wells was sacrificed to quantify cell counts in supernatant and biofilm fraction at the 24 h timepoint. The spent supernatant was gently drained from the rest of the wells and fresh medium or mucin/monosaccharide solutions in medium were added on top of the bacterial biomass.

Plate was gently rocked (100 rpm) for 24 h in the anaerobic atmosphere (generated with AnaeroPack, Mitsubishi in the airtight container) in the 37° C. incubator. After 24 h, the supernatant was removed into a separate plate and the biofilms were washed twice with 1004, sterile phosphate-buffer saline (PBS) and washes were combined with the supernatant (=supernatant fraction). Another 1004, of PBS was added to the biofilms, each well was vigorously scraped with P20 tip for 20 s and scraped biomass pipetted up and down to homogenize the solution (=biofilm fraction). Both supernatant and biofilm solutions were serially diluted, plated on BHI-agar (BHI=brain-heart infusion, 1.5% agar) and incubated in the anaerobic chamber 37° C. incubator. Colonies were counted after ˜48 h.

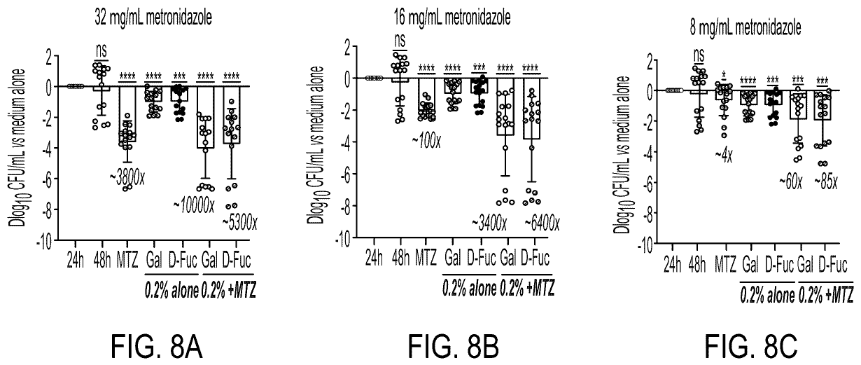

### Galactose and D-Fucose Potentiate the Action of Antibiotic Metronidazole in a Minimum Biofilm Eradication Assay (FIGS. 8A-C)

Metronidazole was purchased from Combi-Blocks. Galactose and D-Fucose were obtained from Sigma-Aldrich.

Mature biofilms of Gardnerella vaginalis were established as described above. On a separate plate serial dilution of metronidazole alone or in combination with 0.2 wt % Gal/D-Fuc were prepared in NYC III 1% Glc medium. Negative controls (no metronidazole) were also included on the plate. After spent supernatant was drained from mature biofilms, 100 μL, of metronidazole/metronidazole-monosaccharide or negative controls solutions were added to the biofilms and plate was rocked (100 rpm) for 24 h in the anaerobic atmosphere (generated with AnaeroPack, Mitsubishi in the airtight container) in the 37° C. incubator. After 24 h incubation, the CFUs of supernatant and biofilm fractions were quantified as described in the biofilm disruption experiment.

### REFERENCES

1. Atashili, J., Poole, C., Ndumbe, P. M., Adimora, A. A. & Smith, J. S. Bacterial vaginosis and HIV acquisition: A meta-analysis of published studies. AIDS Lond. Engl. 22, 1493-1501 (2008).
2. Masao, S. et al. Association between preterm delivery and bacterial vaginosis with or without treatment. Sci. Rep. Nat. Publ. Group Lond. 9, (2019).
3. Catlin, B. W. Gardnerella vaginalis: characteristics, clinical considerations, and controversies. Clin. Microbiol. Rev. 5, 213-237 (1992).
4. Fredricks, D. N., Fiedler, T. L. & Marrazzo, J. M. Molecular Identification of Bacteria Associated with Bacterial Vaginosis. N. Engl. J. Med. 353, 1899-1911 (2005).
5. Information, N. C. for B., Pike, U. S. N. L. of M. 8600 R., MD, B. & Usa, 20894. Which treatments are effective for bacterial vaginosis? InformedHealth. org [Internet] (Institute for Quality and Efficiency in Health Care (IQWiG), 2018).
6. McShane, A. et al. Mucus. Curr. Biol. 31, R938-R945 (2021).
7. van de Wijgert, J. H. H. M. & Jespers, V. The global health impact of vaginal dysbiosis. Res. Microbiol. 168, 859-864 (2017).
8. Koumans, E. H. et al. The Prevalence of Bacterial Vaginosis in the United States, 2001-2004; Associations With Symptoms, Sexual Behaviors, and Reproductive Health. Sex. Transm. Dis. 34, 864-869 (2007).
9. Kenyon, C., Colebunders, R. & Crucitti, T. The global epidemiology of bacterial vaginosis: a systematic review. Am. J. Obstet. Gynecol. 209, 505-523 (2013).
10. Ghartey, J. P. et al. Lactobacillus crispatus Dominant Vaginal Microbiome Is Associated with Inhibitory Activity of Female Genital Tract Secretions against Escherichia coli. PLOS ONE 9, e96659 (2014).
11. Wang, S. et al. Antimicrobial Compounds Produced by Vaginal Lactobacillus crispatus Are Able to Strongly Inhibit Candida albicans Growth, Hyphal Formation and Regulate Virulence-related Gene Expressions. Front. Microbiol. 8, (2017).
12. Breshears, L. M., Edwards, V. L., Ravel, J. & Peterson, M. L. Lactobacillus crispatus inhibits growth of Gardnerella vaginalis and Neisseria gonorrhoeae on a porcine vaginal mucosa model. BMC Microbiol. 15, 276 (2015).
13. Donnarumma, G. et al. Lactobacillus crispatus Ll: high cell density cultivation and exopolysaccharide structure characterization to highlight potentially beneficial effects against vaginal pathogens. BMC Microbiol. 14, 137 (2014).
14. Macklaim, J. M. et al. Comparative meta-RNA-seq of the vaginal microbiota and differential expression by Lactobacillus iners in health and dysbiosis. Microbiome 1, 12 (2013).
15. Jakobsson, T. & Forsum, U. Lactobacillus iners: a Marker of Changes in the Vaginal Flora? J Clin. Microbiol. 45, 3145-3145 (2007).
16. Macklaim, J. M., Gloor, G. B., Anukam, K. C., Cribby, S. & Reid, G. At the crossroads of vaginal health and disease, the genome sequence of Lactobacillus iners AB-1. Proc. Natl. Acad. Sci. U.S.A 108 Suppl 1, 4688-4695 (2011).
17. Randis, T. M. & Ratner, A. J. Gardnerella and Prevotella: Co-conspirators in the Pathogenesis of Bacterial Vaginosis. J. Infect. Dis. 220, 1085-1088 (2019).
18. Polatti, F. Bacterial Vaginosis, Atopobium vaginae and Nifuratel. Curr. Clin. Pharmacol. 7, 36-40 (2012).
19. Bunyan, I., Sarraji, A. & K. Hameed, A. Molecular Detection and Genotyping of Gardnerella Vaginalis, 16S rRNA Gene from Bacterial Vaginosis Miscarriage Women in AL-Hillah City. Int. J Psychosoc. Rehabil. 24, (2020).
20. De Seta, F., Campisciano, G., Zanotta, N., Ricci, G. & Comar, M. The Vaginal Community State Types Microbiome-Immune Network as Key Factor for Bacterial Vaginosis and Aerobic Vaginitis. Front. Microbiol. 10, 2451 (2019).
21. Ma, Z. (Sam) & Li, L. Quantifying the human vaginal community state types (CSTs) with the species specificity index. PeerJ 5, e3366 (2017).
22. Lee, S. et al. Community State Types of Vaginal Microbiota and Four Types of Abnormal Vaginal Microbiota in Pregnant Korean Women. Front. Public Health 8, 612 (2020).
23. Freitas, A. C. & Hill, J. E. Quantification, isolation and characterization of Bifidobacterium from the vaginal microbiomes of reproductive aged women. Anaerobe 47, 145-156 (2017).
24. Aroutcheva, A. A., Simoes, J. A., Behbakht, K. & Faro, S. Gardnerella vaginalis Isolated from Patients with Bacterial Vaginosis and from Patients with Healthy Vaginal Ecosystems. Clin. Infect. Dis. 33, 1022-1027 (2001).
25. Ma, B., Forney, L. J. & Ravel, J. Vaginal Microbiome: Rethinking Health and Disease. Annu. Rev. Microbiol. 66, 371-389 (2012).
26. Jones, A. Bacterial Vaginosis: A Review of Treatment, Recurrence, and Disparities. J. Nurse Pract. 15, 420-423 (2019).
27. Lev-Sagie, A. Vaginal Microbiome Transplantation for Recurrent Bacterial Vaginosis-A Placebo, Randomized, Controlled Trial. https://clinicaltrials.gov/ct2/show/NCT04517487 (2021).
28. Lev-Sagie, A. et al. Vaginal microbiome transplantation in women with intractable bacterial vaginosis. Nat. Med. 25, 1500-1504 (2019).
29. Patterson, J. L., Stull-Lane, A., Girerd, P. H. & Jefferson, K. K. Analysis of adherence, biofilm formation and cytotoxicity suggests a greater virulence potential of Gardnerella vaginalis relative to other bacterial-vaginosis-associated anaerobes. Microbiol. Read. Engl. 156, 392-399 (2010).
30. Gelber, S. E., Aguilar, J. L., Lewis, K. L. T. & Ratner, A. J. Functional and Phylogenetic Characterization of Vaginolysin, the Human-Specific Cytolysin from Gardnerella vaginalis. J. Bacteriol. 190, 3896-3903 (2008).
31. Nowak, R. G. et al. Higher Levels of a Cytotoxic Protein, Vaginolysin, in Lactobacillus-Deficient Community State Types at the Vaginal Mucosa. Sex. Transm. Dis. 45, e14 (2018).
32. Castro, J. et al. Using an in-vitro biofilm model to assess the virulence potential of Bacterial Vaginosis or non-Bacterial Vaginosis Gardnerella vaginalis isolates. Sci. Rep. 5, (2015).
33. Wickstrom, C., Davies, J. R., Eriksen, G. V., Veerman, E. C. & Carlstedt, I. MUC5B is a major gel-forming, oligomeric mucin from human salivary gland, respiratory tract and endocervix: identification of glycoforms and C-terminal cleavage. Biochem. J. 334 (Pt 3), 685-693 (1998).
34. Wang, B. X., Wu, C. M. & Ribbeck, K. Home, sweet home: how mucus accommodates our microbiota. FEBS J. 288, 1789-1799 (2021).
35. Witten, J. & Ribbeck, K. The particle in the spider's web: transport through biological hydrogels. Nanoscale 9, 8080-8095 (2017).
36. Bergstrom, K. et al. Proximal colon-derived O-glycosylated mucus encapsulates and modulates the microbiota. Science 370, 467-472 (2020).
37. Wang, B. X. et al. Mucin Glycans Signal through the Sensor Kinase RetS to Inhibit Virulence-Associated Traits in Pseudomonas aeruginosa. Curr. Biol. 31, 90-102.e7 (2021).
38. Kavanaugh, N. L., Zhang, A. Q., Nobile, C. J., Johnson, A. D. & Ribbeck, K. Mucins Suppress Virulence Traits of Candida albicans. mBio 5, e01911-14 (2014).
39. Wheeler, K. M. et al. Mucin glycans attenuate the virulence of Pseudomonas aeruginosa in infection. Nat. Microbiol. 4, 2146-2154 (2019).
40. Werlang, C. A. et al. Mucin O-glycans suppress quorum-sensing pathways and genetic transformation in Streptococcus mutans. Nat. Microbiol. 1-10 (2021) doi:10.1038/s41564-021-00876-1.
41. Hardy, L. et al. The presence of the putative Gardnerella vaginalis sialidase A gene in vaginal specimens is associated with bacterial vaginosis biofilm. PLOS ONE 12, e0172522 (2017).
42. Kruger, A. G. et al. Stereochemical Control Yields Mucin Mimetic Polymers. ACS Cent. Sci. 7, 624-630 (2021).
43. Adhya, S. & Echols, H. Glucose Effect and the Galactose Enzymes of Escherichia coli: Correlation Between Glucose Inhibition of Induction and Inducer Transport. J. Bacteriol. 92, 601-608 (1966).
44. Buttin, G. Mécanismes régulateurs dans la biosynthèse des enzymes du métabolisme du galactose chez Escherichia coli K12: I. La biosynthèse induite de la galactokinase et l'induction simultanée de la séquence enzymatique. J. Mol. Biol. 7, 164-182 (1963).
45. Magasanik, B. Catabolite repression. Cold Spring Harb. Symp. Quant. Biol. 26, 249-256 (1961).
46. Paixão, L. et al. Host Glycan Sugar-Specific Pathways in Streptococcus pneumonia: Galactose as a Key Sugar in Colonisation and Infection. PLOS ONE 10, e0121042 (2015).
47. Al-Bayati, F. A. Y. et al. Pneumococcal galactose catabolism is controlled by multiple regulators acting on pyruvate formate lyase. Sci. Rep. 7, 43587 (2017).
48. Gaspar, P., Al-Bayati, F. A. Y., Andrew, P. W., Neves, A. R. & Yesilkaya, H. Lactate Dehydrogenase Is the Key Enzyme for Pneumococcal Pyruvate Metabolism and Pneumococcal Survival in Blood. Infect. Immun. 82, 5099-5109 (2014).
49. Yesilkaya, H. et al. Pyruvate formate lyase is required for pneumococcal fermentative metabolism and virulence. Infect. Immun. 77, 5418-5427 (2009).
50. Terra, V. S., Homer, K. A., Rao, S. G., Andrew, P. W. & Yesilkaya, H. Characterization of novel beta-galactosidase activity that contributes to glycoprotein degradation and virulence in Streptococcus pneumoniae. Infect. Immun. 78, 348-357 (2010).
51. Kamaruzzaman, N. F. et al. Antimicrobial Polymers: The Potential Replacement of Existing Antibiotics? Int. J. Mol. Sci. 20, 2747 (2019).
52. Salinas, A. M. et al. Bacterial identification of the vaginal microbiota in Ecuadorian pregnant teenagers: an exploratory analysis. PeerJ 6, e4317 (2018).
53. Frenkel, E. S. & Ribbeck, K. Salivary Mucins Protect Surfaces from Colonization by Cariogenic Bacteria. Appl. Environ. Microbiol. 81, 332-338 (2015).
54. Sahoo, J. K. et al. Sugar Functionalization of Silks with Pathway-Controlled Substitution and Properties. Adv. Biol. 5, e2100388 (2021).
55. Frenkel, E. S. & Ribbeck, K. Salivary Mucins Protect Surfaces from Colonization by Cariogenic Bacteria. Appl. Environ. Microbiol. 81, 332-338 (2015).
56. Percec, V. et. al. Modular Synthesis of Amphiphilic Janus Glycodendrimers and Their Self-Assembly into Glycodendrimersomes and Other Complex Architectures with Bioactivity to Biomedically Relevant Lectins. J. Am. Chem. Soc. 135 (24), 9055-9077 (2013).
57. Werther, P.; Möhler, J. S.; Wombacher, R. A Bifunctional Fluorogenic Rhodamine Probe for Proximity-Induced Bioorthogonal Chemistry. Chem.—A Eur. J. 23 (72), 1821618224 (2017).
58. Yan, T et al. Synthesis of Tungsten Oxo Alkylidene Biphenolate Complexes and Ring-Opening Metathesis Polymerization of Norbornenes and Norbornadienes. Organometallics 38 (16), 3144-3150 (2019).

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

It should be understood that the subject matter defined in the appended claims is not necessarily limited to the specific implementations described above.

1. A method of treating or preventing a vaginal infection of Gardnerella vaginalis in a subject, the method comprising administering to the subject an effective amount of a composition comprising one or more of galactose, D-fucose, or mucin 5B.

2. The method of claim 1, wherein the composition comprises galactose.

3. The method of claim 1, wherein composition comprises D-fucose.

4. The method of claim 1, wherein the composition comprises mucin 5B.

5. The method of claim 1, further comprising diagnosing the subject with the vaginal infection of Gardnerella vaginalis.

6. The method of claim 1, wherein the method reduces concentration of Gardnerella vaginalis in the vagina of the subject.

7. The method of claim 1, wherein the method reduces expression of vaginolysin by Gardnerella vaginalis in the vagina of the subject.

8. The method of claim 1, wherein the method reduces concentration of vaginolysin in the vagina of the subject.

9. The method of claim 1, wherein the method reduces mortality rates for endocervical cells of the subject.

10. The method of claim 1, wherein the method reduces biofilm formation caused by the vaginal infection of Gardnerella vaginalis.

11. The method of claim 1, wherein the method treats the vaginal infection of Gardnerella vaginalis.

12. The method of claim 1, wherein the method prevents the vaginal infection of Gardnerella vaginalis.

13. The method of claim 1, wherein the subject has bacterial vaginosis.

14. The method of claim 1, wherein the subject has previously had at least three bacterial vaginosis infections.

15. The method of claim 1, wherein the galactose, D-fucose, or mucin 5B is administered in an amount from 0.1 wt % to 50 wt %.

16. The method of claim 1, wherein the composition is formulated for topical administration.

17. The method of claim 1, wherein the composition is formulated for vaginal administration.

18. The method of claim 1, further comprising administering a Lactobacillus probiotic to the subject.

19. The method of claim 1, further comprising administering an antibiotic to the subject.

20. The method of claim 19, wherein the antibiotic is metronidazole.

21. A method of treating or preventing a vaginal infection of Gardnerella vaginalis in a subject, the method comprising administering to the subject an effective amount of a composition comprising one or more of galactose, N-acetyl D-galactosamine, N-acetyl D-glucosamine, D-fucose, or mucin 5B bonded to a surface.

22. The method of claim 21, wherein the composition comprises galactose bonded to the surface.

23. The method of claim 21, wherein the composition comprises N-acetyl D-galactosamine bonded to the surface.

24. The method of claim 21, wherein the composition comprises N-acetyl D-glucosamine bonded to the surface.

25. The method of claim 21, wherein the composition comprises D-fucose bonded to the surface.

26. The method of claim 21, wherein the composition comprises mucin 5B bonded to the surface.

27. The method of claim 21, wherein the surface is a polymer.

28. The method of claim 21, wherein the surface is a silk fibroin (SF).

29. The method of claim 21, wherein the surface is a norbornene polymer.

30. The method of claim 21, wherein the surface is a star polymer.

31. The method of claim 21, wherein the surface is a dendrimer.

32-47. (canceled)
